SEQUENCE LISTING (37 sequences)

SEQ ID NO: 1 - protein, 198 aa, Homo sapiens
MAKQPSDVSSECDREGRQLQPAERPPQLRPGAPTSLQTEPQGNPEGNHGGEGDSCPHGSPQGPLAPPASPGPFATRSPLFIFMRRSSLLSRSSSGYFSFDTDRSPAPMSCDKSTQTPSPPCQAFNHYLSAMASMRQAEPADMRPEIWIAQELRRIGDEFNAYYARRVFLNNYQAAEDHPRMVILRLLRYIVRLVWRMH

SEQ ID NO: 2 - protein, 138 aa, Homo sapiens
MAKQPSDVSSECDREGRQLQPAERPPQLRPGAPTSLQTEPQDRSPAPMSCDKSTQTPSPPCQAFNHYLSAMASMRQAEPADMRPEIWIAQELRRIGDEFNAYYARRVFLNNYQAAEDHPRMVILRLLRYIVRLVWRMH

SEQ ID NO: 3 - protein, 108 aa, Homo sapiens
MAKQPSDVSSECDREGRQLQPAERPPQLRPGAPTSLQTEPQASMRQAEPADMRPEIWIAQELRRIGDEFNAYYARRVFLNNYQAAEDHPRMVILRLLRYIVRLVWRMH

SEQ ID NO: 4 - protein, 193 aa, Homo sapiens
MARARQEGSSPEPVEGLARDGPRPFPLGRLVPSAVSCGLCEPGLAAAPAAPTLLPAAYLCAPTAPPAVTAALGGSRWPGGPRSRPRGPRPDGPQPSLSLAEQHLESPVPSAPGALAGGPTQAAPGVRGEEEQWAREIGAQLRRMADDLNAQYERRRQEEQQRHRPSPWRVLYNLIMGLLPLPRGHRAPEMEPN

SEQ ID NO: 5 - protein, 131 aa, Homo sapiens
MKFGMGSAQACPCQVPRAASTTWVPCQICGPQPSLSLAEQHLESPVPSAPGALAGGPTQAAPGVRGEEEQWAREIGAQLRRMADDLNAQYERRRQEEQQRHRPSPWRVLYNLIMGLLPLPRGHRAPEMEPN

SEQ ID NO: 6 - protein, 101 aa, Homo sapiens
MKFGMGSAQACPCQVPRAASTTWVPCQICETRGAAAAPPLTLEGPVQSHHGTPALTQGPQSPRDGAQLGACTRPVDVRDSGGRPLPPPDTLASAGDFLCTM

SEQ ID NO: 7 - protein, 192 aa, Homo sapiens
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 8 - protein, 218 aa, Homo sapiens
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWVRLLKPPHPHHRALTTAPAPPSLPPATPLGPWAFWSRSQWCPLPIFRSSDVVYNAFSLRV

SEQ ID NO: 9 - protein, 143 aa, Homo sapiens
MDGSGEQPRGGGPTSSEQIMKTGALLLQGMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 10 - protein, 164 aa, Homo sapiens
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKAGVKWRDLGSLQPLPPGFKRFTCLSIPRSWDYRPCAPRCRN

SEQ ID NO: 11 - protein, 179 aa, Homo sapiens
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWTVTIFVAGVLTASLTIWKKMG

SEQ ID NO: 12 - protein, 173 aa, Homo sapiens
MKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 13 - protein, 114 aa, Homo sapiens
MIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 14 - RNA, 597 nt, Homo sapiens
auggcaaagcaaccuucugauguaaguucugagugugaccgagaagguagacaauugcagccugcggagaggccuccccagcucagaccuggggccccuaccucccuacagacagagccacaagguaauccugaaggcaaucacggaggugaaggggacagcugcccccacggcagcccucagggcccgcuggccccaccugccagcccuggcccuuuugcuaccagauccccgcuuuucaucuuuaugagaagauccucccugcugucucgauccuccaguggguauuucucuuuugacacagacaggagcccagcacccaugaguugugacaaaucaacacaaaccccaaguccuccuugccaggccuucaaccacuaucucagugcaauggcuuccaugaggcaggcugaaccugcagauaugcgcccagagauauggaucgcccaagaguugcggcguaucggagacgaguuuaacgcuuacuaugcaaggaggguauuuuugaauaauuaccaagcagccgaagaccacccacgaaugguuaucuuacgacuguuacguuacauuguccgccugguguggagaaugcauuga

SEQ ID NO: 15 - RNA, 417 nt, Homo sapiens
auggcaaagcaaccuucugauguaaguucugagugugaccgagaagguagacaauugcagccugcggagaggccuccccagcucagaccuggggccccuaccucccuacagacagagccacaagacaggagcccagcacccaugaguugugacaaaucaacacaaaccccaaguccuccuugccaggccuucaaccacuaucucagugcaauggcuuccaugaggcaggcugaaccugcagauaugcgcccagagauauggaucgcccaagaguugcggcguaucggagacgaguuuaacgcuuacuaugcaaggaggguauuuuugaauaauuaccaagcagccgaagaccacccacgaaugguuaucuuacgacuguuacguuacauuguccgccugguguggagaaugcauuga

SEQ ID NO: 16 - RNA, 327 nt, Homo sapiens
auggcaaagcaaccuucugauguaaguucugagugugaccgagaagguagacaauugcagccugcggagaggccuccccagcucagaccuggggccccuaccucccuacagacagagccacaagcuuccaugaggcaggcugaaccugcagauaugcgcccagagauauggaucgcccaagaguugcggcguaucggagacgaguuuaacgcuuacuaugcaaggaggguauuuuugaauaauuaccaagcagccgaagaccacccacgaaugguuaucuuacgacuguuacguuacauuguccgccugguguggagaaugcauuga

SEQ ID NO: 17 - RNA, 582 nt, Homo sapiens
auggcccgcgcacgccaggagggcagcuccccggagcccguagagggccuggcccgcgacggcccgcgccccuucccgcucggccgccuggugcccucggcaguguccugcggccucugcgagcccggccuggcugccgcccccgccgcccccacccugcugcccgcugccuaccucugcgcccccaccgccccacccgccgucaccgccgcccugggggguucccgcuggccuggggguccccgcagccggccccgaggcccgcgcccggacgguccucagcccucgcucucgcuggcggagcagcaccuggagucgcccgugcccagcgccccgggggcucuggcgggcggucccacccaggcggccccgggaguccgcggggaggaggaacagugggcccgggagaucggggcccagcugcggcggauggcggacgaccucaacgcacaguacgagcggcggagacaagaggagcagcagcggcaccgccccucacccuggaggguccuguacaaucucaucaugggacuccugcccuuacccaggggccacagagcccccgagauggagcccaauuag

SEQ ID NO: 18 - RNA, 396 nt, Homo sapiens
augaaauuuggcauggggucugcccaggcauguccaugccaggugcccagggcugcuuccacgacguggguccccugccagauuugugguccucagcccucgcucucgcuggcggagcagcaccuggagucgcccgugcccagcgccccgggggcucuggcgggcggucccacccaggcggccccgggaguccgcggggaggaggaacagugggcccgggagaucggggcccagcugcggcggauggcggacgaccucaacgcacaguacgagcggcggagacaagaggagcagcagcggcaccgccccucacccuggaggguccuguacaaucucaucaugggacuccugcccuuacccaggggccacagagcccccgagauggagcccaauuag

SEQ ID NO: 19 - RNA, 306 nt, Homo sapiens
augaaauuuggcauggggucugcccaggcauguccaugccaggugcccagggcugcuuccacgacguggguccccugccagauuugugagacaagaggagcagcagcggcaccgccccucacccuggaggguccuguacaaucucaucaugggacuccugcccuuacccaggggccacagagcccccgagauggagcccaauuaggugccugcacccgcccgguggacgucagggacucggggggcaggccccucccaccuccugacacccuggccagcgcgggggacuuucucugcaccauguag

SEQ ID NO: 20 - RNA, 579 nt, Homo sapiens
auggacggguccggggagcagcccagaggcggggggcccaccagcucugagcagaucaugaagacaggggcccuuuugcuucaggguuucauccaggaucgagcagggcgaauggggggggaggcacccgagcuggcccuggacccggugccucaggaugcguccaccaagaagcugagcgagugucucaagcgcaucggggacgaacuggacaguaacauggagcugcagaggaugauugccgccguggacacagacuccccccgagaggucuuuuuccgaguggcagcugacauguuuucugacggcaacuucaacuggggccggguugucgcccuuuucuacuuugccagcaaacuggugcucaaggcccugugcaccaaggugccggaacugaucagaaccaucaugggcuggacauuggacuuccuccgggagcggcuguugggcuggauccaagaccagggugguugggacggccuccucuccuacuuugggacgcccacguggcagaccgugaccaucuuuguggcgggagugcucaccgccucacucaccaucuggaagaagaugggcuga

SEQ ID NO: 21 - RNA, 657 nt, Homo sapiens
auggacggguccggggagcagcccagaggcggggggcccaccagcucugagcagaucaugaagacaggggcccuuuugcuucaggguuucauccaggaucgagcagggcgaauggggggggaggcacccgagcuggcccuggacccggugccucaggaugcguccaccaagaagcugagcgagugucucaagcgcaucggggacgaacuggacaguaacauggagcugcagaggaugauugccgccguggacacagacuccccccgagaggucuuuuuccgaguggcagcugacauguuuucugacggcaacuucaacuggggccggguugucgcccuuuucuacuuugccagcaaacuggugcucaaggcccugugcaccaaggugccggaacugaucagaaccaucaugggcuggacauuggacuuccuccgggagcggcuguugggcuggauccaagaccagggugguugggugagacuccucaagccuccucacccccaccaccgcgcccucaccaccgccccugccccaccgucccugccccccgccacuccucugggacccugggccuucuggagcaggucacaguggugcccucuccccaucuucagaucaucagauguggucuauaaugcguuuuccuuacgugucuga

SEQ ID NO: 22 - RNA, 432 nt, Homo sapiens
auggacggguccggggagcagcccagaggcggggggcccaccagcucugagcagaucaugaagacaggggcccuuuugcuucaggggaugauugccgccguggacacagacuccccccgagaggucuuuuuccgaguggcagcugacauguuuucugacggcaacuucaacuggggccggguugucgcccuuuucuacuuugccagcaaacuggugcucaaggcccugugcaccaaggugccggaacugaucagaaccaucaugggcuggacauuggacuuccuccgggagcggcuguugggcuggauccaagaccagggugguugggacggccuccucuccuacuuugggacgcccacguggcagaccgugaccaucuuuguggcgggagugcucaccgccucgcucaccaucuggaagaagaugggcuga

SEQ ID NO: 23 - RNA, 495 nt, Homo sapiens
auggacggguccggggagcagcccagaggcggggggcccaccagcucugagcagaucaugaagacaggggcccuuuugcuucaggguuucauccaggaucgagcagggcgaauggggggggaggcacccgagcuggcccuggacccggugccucaggaugcguccaccaagaagcugagcgagugucucaagcgcaucggggacgaacuggacaguaacauggagcugcagaggaugauugccgccguggacacagacuccccccgagaggucuuuuuccgaguggcagcugacauguuuucugacggcaacuucaacuggggccggguugucgcccuuuucuacuuugccagcaaacuggugcucaaggcuggcgugaaauggcgugaucugggcucacugcaaccucugccuccuggguucaagcgauucaccugccucagcaucccaaggagcugggauuacaggcccugugcaccaaggugccggaacuga

SEQ ID NO: 24 - RNA, 540 nt, Homo sapiens
auggacggguccggggagcagcccagaggcggggggcccaccagcucugagcagaucaugaagacaggggcccuuuugcuucaggguuucauccaggaucgagcagggcgaauggggggggaggcacccgagcuggcccuggacccggugccucaggaugcguccaccaagaagcugagcgagugucucaagcgcaucggggacgaacuggacaguaacauggagcugcagaggaugauugccgccguggacacagacuccccccgagaggucuuuuuccgaguggcagcugacauguuuucugacggcaacuucaacuggggccggguugucgcccuuuucuacuuugccagcaaacuggugcucaaggcccugugcaccaaggugccggaacugaucagaaccaucaugggcuggacauuggacuuccuccgggagcggcuguugggcuggauccaagaccagggugguuggaccgugaccaucuuuguggcgggagugcucaccgccucacucaccaucuggaagaagaugggcuga

SEQ ID NO: 25 - RNA, 522 nt, Homo sapiens
augaagacaggggcccuuuugcuucaggguuucauccaggaucgagcagggcgaauggggggggaggcacccgagcuggcccuggacccggugccucaggaugcguccaccaagaagcugagcgagugucucaagcgcaucggggacgaacuggacaguaacauggagcugcagaggaugauugccgccguggacacagacuccccccgagaggucuuuuuccgaguggcagcugacauguuuucugacggcaacuucaacuggggccggguugucgcccuuuucuacuuugccagcaaacuggugcucaaggcccugugcaccaaggugccggaacugaucagaaccaucaugggcuggacauuggacuuccuccgggagcggcuguugggcuggauccaagaccagggugguugggacggccuccucuccuacuuugggacgcccacguggcagaccgugaccaucuuuguggcgggagugcucaccgccucacucaccaucuggaagaagaugggcuga

SEQ ID NO: 26 - RNA, 345 nt, Homo sapiens
augauugccgccguggacacagacuccccccgagaggucuuuuuccgaguggcagcugacauguuuucugacggcaacuucaacuggggccggguugucgcccuuuucuacuuugccagcaaacuggugcucaaggcccugugcaccaaggugccggaacugaucagaaccaucaugggcuggacauuggacuuccuccgggagcggcuguugggcuggauccaagaccagggugguugggacggccuccucuccuacuuugggacgcccacguggcagaccgugaccaucuuuguggcgggagugcucaccgccucacucaccaucuggaagaagaugggcuga

SEQ ID NO: 27 - protein, 12 aa, Artificial Sequence
  Cell-penetrating motif
AAVLLPVLLAAP

SEQ ID NO: 28 - DNA, 25 bp, Artificial Sequence
  Oligonucleotide primer
ctctcatcggagatgatattaaccg

SEQ ID NO: 29 - DNA, 25 bp, Artificial Sequence
  Oligonucleotide primer
agtatgatatcagccaaaaagcagg

SEQ ID NO: 30 - DNA, 25 bp, Artificial Sequence
  Oligonucleotide primer
ggagatgaactggatagcaatatgg

SEQ ID NO: 31 - DNA, 25 bp, Artificial Sequence
  Oligonucleotide primer
gtttgctagcaaagtagaagagggc

SEQ ID NO: 32 - DNA, 25 bp, Artificial Sequence
  Oligonucleotide primer
gagttgtgacaagtcaacacaaacc

SEQ ID NO: 33 - DNA, 31 bp, Artificial Sequence
  Oligonucleotide primer
gaagataaagcgtaacagttgtaagataacc

SEQ ID NO: 34 - DNA, 21 bp, Artificial Sequence
  Oligonucleotide primer
atgcctgcctcaccttcatct

SEQ ID NO: 35 - DNA, 22 bp, Artificial Sequence
  Oligonucleotide primer
agcacaggattcacagtctgga

SEQ ID NO: 36 - DNA, 19 bp, Artificial Sequence
  Oligonucleotide primer
tattggcaacgagcggttc

SEQ ID NO: 37 - DNA, 21 bp, Artificial Sequence
  Oligonucleotide primer
ccatacccaagaaggaaggct

(19) United States
(12) Patent Application Publication
(10) Pub. No.: US 2014/0072567 A1
(43) Pub. Date: Mar. 13, 2014

(54) Methods Of Treating And Preventing Glucose Toxicity

(71) Applicants: Walter and Eliza Hall Institute of Medical Research, ACN 004 251 423, Parkville (AU); St. Vincent's Institute of Medical Research, Can 004 705 640, Fitzroy (AU)

(72) Inventors: Andreas Strasser, Ascot Vale (AU); Mark McKenzie, Sunbury (AU); Helen Thomas, Carlton (AU); Janette Allison, Essendon (AU); Tom Kay, Kew (AU)

(73) Assignees: Walter and Eliza Hall Institute of Medical Research, ACN 004 251 423, Parkville (AU); St. Vincent's Institute of Medical Research, CAN 004 705 640, Fitzroy (AU)

(21) Appl. No.: 13/972,476

(22) Filed: Aug. 21, 2013

Related U.S. Application Data
(63) Continuation of application No. 13/021,279, filed on Feb. 4, 2011.
Application No. 13/021,279 is a continuation-in-part of application No. PCT/AU2009/001003, filed on Aug. 5, 2009.
(60) Provisional application No. 61/086,759, filed on Aug. 6, 2008.

(51) Int. Cl. C12N 15/113; C07K 16/22

(52) U.S. Cl. CPC C12N 15/1135; C07K 16/22
USPC 424/139.1; 514/44 A

(57) ABSTRACT

The present invention relates to a method for treating and/or preventing a disease associated with glucose toxicity in a subject which may comprise administering to the subject a compound that reduces the level of Bim, PUMA and/or Bax activity in a cell of the subject. Furthermore, provided is a method for culturing cells susceptible to exposure to high concentrations of glucose, which may comprise delivering to the cells, or progenitor cells thereof, a compound that reduces the level of Bim, PUMA and/or Bax activity in the cells.

### RELATED APPLICATIONS AND INCORPORATION BY REFERENCE

This application is a continuation application of U.S. patent application Ser. No. 13/021,279 filed 4 Feb. 2011, which is a continuation-in-part application of international patent application Serial No. PCT/AU2009/001003 filed 5 Aug. 2009, which published as PCT Publication No. WO 2010/015032 on 11 Feb. 2010, which claims benefit of U.S. provisional patent application Ser. No. 61/086,756 filed 6 Aug. 2008.

The foregoing applications, and all documents cited therein or during their prosecution (“appin cited documents”) and all documents cited or referenced in the appin cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to a method for treating and/or preventing a disease associated with glucose toxicity in a subject which may comprise administering to the subject a compound that reduces the level of Bim, PUMA and/or Bax activity in a cell of the subject. Furthermore, provided is a method for culturing cells susceptible to exposure to high concentrations of glucose, which may comprise delivering to the cells, or progenitor cells thereof, a compound that reduces the level of Bim, PUMA and/or Bax activity in the cells.

### BACKGROUND OF THE INVENTION

It is increasingly recognised in both animals and humans that type 2 diabetes (T2D) only develops when insulin resistant subjects develop pancreatic beta cell dysfunction (Rhodes, 2005; Prentki and Nolan, 2006; Muoio and Newgard, 2008). Progressive beta cell dysfunction results in beta cells failing to secrete sufficient amounts of insulin to compensate for insulin resistance. The relative contribution of a decrease in beta cell mass (due in part to abnormal beta cell death) versus a defect in insulin secretion has been controversial. Butler et al. (2003), using human pancreatic tissue from autopsies, showed that there was a 60% reduction in beta cell mass in T2D patients compared to non-diabetic controls, that was attributed to a 10-fold or 3-fold increase in beta cell apoptosis in T2D patients who were lean or obese, respectively.

Several mediators that underlie the increased rate of apoptosis have been proposed, including exposure to high concentrations of glucose (termed “glucotoxicity”), lipids, free fatty acids, leptin, pro-inflammatory cytokines (for example, TNFα, IL-6, IL-1β) and islet cell amyloid. Amongst these, an increased concentration of glucose is believed to be the major factor contributing to beta cell killing. It has been suggested that glucotoxicity induces beta-cell apoptosis in human islets by beta cell intrinsic production of IL-1β, NFκB activation, Fas up-regulation and beta cell apoptosis by engagement of Fas by Fas ligand produced on neighbouring cells (Maedler et al., 2001; Maedler et al., 2002). However these ideas remain controversial as another group failed to reproduce the key findings of IL-1β production and NFκB activation in a study of human islet exposure to high concentrations of glucose, using both non-diabetic as well as diabetic donors (Welsh et al., 2005).

Several other studies have suggested that glucose may induce endoplasmic reticulum (ER) stress in beta cells (and possibly also other cell types that are affected by gluco-toxicity in type 2 diabetes; reviewed by Eizirik et al., 2008). Beta cells are particularly vulnerable to ER stress due to their enormous demand to synthesize and secrete insulin. Oxidative stress has also been implicated in glucose toxicity (reviewed by Robertson, 2004). Intrinsic anti-oxidant enzyme expression in islets is low (Tiedge et al., 1997), and high glucose can induce reactive oxygen species (ROS) production. It has also been reported that anti-oxidant drugs or over-expression of anti-oxidant enzymes (Tanaka et al., 2002; Robertson, 2007) can block glucose toxicity.

In mammalian cells two distinct pathways control apoptosis: the “death receptor” (also called extrinsic) and the “mitochondrial” (also called “intrinsic” or “Bcl-2 regulated”) pathways. The “death receptor” pathway results from engagement of members of the tumor necrosis factor receptor (TNF-R) family that have an intra-cellular ‘death domain’ (e.g. Fas, TNF-R1) that signal apoptosis by direct activation of the caspase cascade. The mitochondrial pathway is activated by developmental cues, growth factor withdrawal and a broad range of cytotoxic stimuli (e.g. chemotherapeutic drugs) and is controlled by pro- and anti-apoptotic members of the Bcl-2 family of proteins (Strasser, 2005). The eight different mammalian BH3-only proteins promote apoptosis by binding to pro-survival Bcl-2 family members, thereby unleashing Bax and/or Bak to promote mitochondrial outer membrane permeabilization and activation of the caspase cascade (Willis et al., 2007) and some of them have also been reported to be able to activate Bax and Bak directly (Letai, 2008).

There is a need for the identification of methods for treating and preventing diseases associated with glucose toxicity such as Type II diabetes.

Citation or identification of any document in this application is not an admission that such document is available as prior art to the present invention.

### SUMMARY OF THE INVENTION

The present inventors have found that Bim, PUMA and Bax are involved in cell death following exposure of a cell, such as a pancreatic β cell, to high concentrations of sugars such as glucose.

Thus, in a first aspect the present invention provides a method for treating and/or preventing a disease associated with glucose toxicity in a subject, the method may comprise administering to the subject a compound that reduces the level of Bim, PUMA and/or Bax activity in a cell of the subject.

In an embodiment, the disease may be Type 2 diabetes, Type 1 diabetes, diabetic neuropathy, diabetic nephropathy or diabetic retinopathy. In a particularly preferred embodiment, the disease may be Type 2 diabetes.

In an embodiment, the compound may bind Bim, PUMA and/or Bax.

In one embodiment, the compound may be a small molecule.

In another embodiment, the compound may be a polypeptide.

In a further embodiment, the polypeptide may be an antibody or an antigenic binding fragment thereof. In yet another embodiment, the antibody or antigenic binding fragment thereof may be an internalizing antibody.

In an embodiment, the compound may be a fragment of Bcl-2.

In an alternate embodiment, the compound may reduce transcription and/or translation of a gene encoding Bim, PUMA and/or Bax.

Preferably, the compound may reduce transcription and/or translation of a gene encoding Bim, PUMA and/or Bax is a polynucleotide. Examples of such polynucleotides include, but are not limited to, an antisense polynucleotide, a sense polynucleotide, a catalytic polynucleotide, a microRNA, and a double-stranded RNA.

In one embodiment, the polynucleotide may be an antisense polynucleotide which hybridises under physiological conditions to a polynucleotide which may comprise any one or more of the sequence of nucleotides provided as SEQ ID NOs 14 to 26.

In another embodiment, the polynucleotide may be a catalytic polynucleotide capable of cleaving, under physiological conditions, a polynucleotide which may comprise any one or more of the sequence of nucleotides provided as SEQ ID NOs 14 to 26. In an embodiment, the catalytic polynucleotide may be a ribozyme.

In a particularly preferred embodiment, the polynucleotide may be a double-stranded RNA (dsRNA) molecule which may comprise an oligonucleotide which may comprise at least 19 contiguous nucleotides of any one or more of the sequence of nucleotides provided as SEQ ID NOs 14 to 26, wherein the portion of the molecule that is double-stranded may be at least 19 basepairs in length and may comprise said oligonucleotide.

In a further embodiment, the dsRNA may be expressed from a single promoter, wherein the strands of the double-stranded portion may be linked by a single-stranded portion.

The compound that may reduce the level of Bim, PUMA and/or Bax activity does not necessarily have to be administered directly to the subject. In an alternate procedure, the compound may be delivered to cells in vitro which are then transplanted into the subject. Thus, in another aspect the present invention provides a method for treating and/or preventing a disease associated with glucose toxicity in a subject, the method which may comprise
i) administering cells in vitro with a compound that reduces the level of Bim, PUMA and/or Bax activity in a cell, and
ii) administering the cells from step i) into the subject.

In a preferred embodiment, the cells may be pancreatic β cells, Vasa nervorum cells, proximal tubular epithelial cells, renal glomerulus cells, renal mesangial cells, retinal capillary cells, or progenitor cells of any one or more thereof. In a particularly preferred embodiment, the cells may be pancreatic β cells, or progenitor cells thereof.

Preferably, the method may further comprise culturing the cells before they are administered to the subject.

In one embodiment, Bim, PUMA and Bax activity may be reduced.

In another embodiment, PUMA and Bax activity may be reduced.

In another embodiment, Bim and Bax activity may be reduced.

In a particularly preferred embodiment, Bim and PUMA activity may be reduced.

When the activity of two or more of Bim, PUMA and Bax may be reduced, the subject or cell may typically be administered with different compounds which specifically may reduce the level of activity and/or each protein in the cell.

In a preferred embodiment, the subject or cell may be administered with a first dsRNA molecule that may reduce the transcription and/or translation of the gene encoding Bim, and a second dsRNA molecule that may reduce the transcription and/or translation of the gene encoding PUMA.

Also provided is the use of a compound that may reduce the level of Bim, PUMA and/or Bax activity in a cell for the manufacture of a medicament for treating and/or preventing a disease associated with glucose toxicity.

Further provided is the use of a compound that may reduce the level of Bim, PUMA and/or Bax activity in a cell as a medicament for treating and/or preventing a disease associated with glucose toxicity.

Compounds that reduce the level of Bim, PUMA and/or Bax activity may also be used to enhance the survival of cells in culture. Thus, in another aspect the present invention provides a method for culturing cells susceptible to exposure to high concentrations of glucose, the method which may comprise delivering to the cells, or progenitor cells thereof, a compound that reduces the level of Bim, PUMA and/or Bax activity in the cells.

In a preferred embodiment, the cells may be cultured in the presence of a high sugar concentration.

In a further aspect, the present invention provides a cell comprising an exogenous compound that may reduce the level of Bim, PUMA and/or Bax activity, wherein an isogenic cell lacking said exogenous compound is susceptible to glucose toxicity.

In a preferred embodiment, the cell may be a pancreatic β cell, a Vasa nervorum cell, a proximal tubular epithelial cell, a renal glomerulus cell, a renal mesangial cell, a retinal capillary cell, or a progenitor cell of any one or more thereof. In a particularly preferred embodiment, the cell may be a pancreatic β cell, or progenitor cell thereof.

In a preferred embodiment, the compound may be an exogenous polynucleotide that reduces transcription and/or translation of a gene encoding Bim, PUMA and/or Bax.

In an embodiment, the cell may be in vitro. In an alternate embodiment, the cell may be in vivo.

In yet a further aspect, the present invention provides a method of identifying a compound for treating and/or preventing a disease associated with glucose toxicity, the method which may comprise
i) identifying a compound which binds Bim, PUMA and/or Bax,
ii) determining if the compound reduces the level of Bim, PUMA and/or Bax activity in a cell.

In another aspect, the present invention provides a method of identifying a compound for treating and/or preventing a disease associated with glucose toxicity, the method which may comprise
i) identifying a compound which hybridizes to a gene encoding Bim, PUMA and/or Bax, or a transcription product thereof, and
ii) determining if the compound reduces the production of Bim, PUMA and/or Bax in a cell,
   wherein the compound may be a polynucleotide.

In a further aspect, the present invention provides a method of identifying a compound for treating and/or preventing a disease associated with glucose toxicity, the method which may comprise
i) identifying a compound comprising an oligonucleotide which comprises at least 19 contiguous nucleotides of any one or more of the sequence of nucleotides provided as SEQ ID NOs 14 to 26, wherein the compound comprises a double-stranded portion that is at least 19 basepairs in length which comprises said oligonucleotide, and
ii) determining if the compound reduces the production of Bim, PUMA and/or Bax in a cell,
   wherein the compound may be a polynucleotide.

Preferably, the screening methods outlined above further comprise delivering the compound to a cell susceptible to glucose toxicity, or progenitor cell thereof, and determining if the compound enhances the survival of the cell, or progeny thereof, when compared to an isogenic cell which has not been exposed to the compound.

In a preferred embodiment, the cell may be a pancreatic βcell, a Vasa nervorum cell, a proximal tubular epithelial cell, a renal glomerulus cell, a renal mesangial cell, a retinal capillary cell, or a progenitor cell of any one or more thereof. In a particularly preferred embodiment, the cell may be a pancreatic β cell, or progenitor cell thereof.

Preferably, survival of the cell may be assessed in the presence of a high sugar concentration. Examples of such sugars include, but are not limited to, glucose, ribose, fructose or a combination thereof.

As will be apparent, preferred features and characteristics of one aspect of the invention are applicable to many other aspects of the invention.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description. The invention is also described by way of the following non-limiting Examples and with reference to the accompanying figures.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1: Wild-type islets cultured in high concentrations of glucose or ribose undergo DNA fragmentation and mitochondrial release of cytochrome C, both indicative of apoptosis, and have a reduced ability to secrete insulin. (A) 200 islets from wild-type C57BL/6 mice were either cultured in low glucose medium (5.6 mM) (unt) or cultured in high glucose media (33.3 mM) for 6 days or high ribose medium (50 mM) for 4 days and DNA fragmentation was measured. Representative flow cytometry profiles are shown i). Data represent islets from 13-17 individual mice per group ii). Statistical significance; *p<0.001 compared with unt (one-way ANOVA). (B) 100 islets from wild-type mice were cultured under conditions mentioned above for 4 and 3 days respectively and cytochrome C release was measured. Representative flow cytometry profiles are shown i). Data represent islets from 4 individual mice per group. ii) Statistical significance; *p<0.001 ** p<0.05 compared with unt (one-way ANOVA). (C) wild-type islets were cultured in high concentrations of ribose (50 mM) for 2 days and insulin release was measured after glucose stimulation. Statistical significance; *p<0.01 compared with unt (one-way ANOVA).

FIG. 2: IL-1β or Fas are not required for glucose or ribose induced beta cell apoptosis. (A) 200 islets from wild-type, IL-1R^{-/-} and B6^{lpr/lpr} mice were cultured for 6 days in 5.6 mM low glucose medium (unt) or 33.3 mM D-glucose or for 4 days in 50 mM ribose. DNA fragmentation was measured by flow cytometry. (B) 100 wild-type islets were cultured for 7 days in 5.6 mM or 33.3 mM D-glucose with cytokines added in the last 4 days and iNOS expression, NO production and DNA fragmentation was measured. Representative Western blot for iNOS expression (β-actin used as loading control). (C) NO₂ determination of culture supernatant (D) DNA fragmentation was measured by flow cytometry. (E) Wild-type islets were cultured in 50 mM ribose or cytokines for 2 days, 33.3 mM D-glucose for 4 days (solid lines) compared to islets cultured in 5.6 mM D-glucose (broken lines). Fas expression on β cells was analysed by FACS analysis. (F) Wild-type islets cultured for 7 days in 5.6 mM (−) or 33.3 mM (+) glucose with cytokines or FasL added in the last 4 days. The data represent the mean±S.E.M of three experiments.

FIG. 3: Over-expression of Bcl-2 in β cells inhibits mitochondrial cytochrome c release and DNA fragmentation under high ribose (50 mM) and high glucose (33.3 mM) conditions. 100 islets from wild-type or Hom.RIP.Bcl-2 mice were (A) untreated or cultured in 50 mM ribose for 3 days and the amount of cytochrome c release was measured. Data represent islets from 5 individual mice per group. Statistical significance; *p<0.001 compared with treated wild-type (wt) (one-way ANOVA). (B) untreated or cultured in 50 mM ribose for 4 days and the amount of DNA fragmentation was measured by flow cytometry Data represent islets from 5-7 individual mice per group. Statistical significance; **p<0.01 compared with treated wt (one-way ANOVA). (C) untreated or cultured in 33.3 mM glucose for 6 days and the amount of DNA fragmentation was measured by flow cytometry. Data represent islets from 4-6 individual mice per group. Statistical significance; #p<0.05 compared with treated wt (one-way ANOVA).

FIG. 4: Loss of Bim or Puma protects islets from glucose- or ribose-induced mitochondrial cytochrome c release and DNA fragmentation. 100 islets from wild-type, bim^{-/-}, noxa^{-/-}, puma^{-/-} or bid^{-/-} mice were cultured in control medium or medium containing 50 mM ribose. (A) Cytochrome c release was measured by flow cytometry after 3 days of culture. Data represent islets from 3-13 individual mice per genotype. Statistical significance; *p<0.0001 compared with wild-type islets in 50 mM ribose (one-way ANOVA). (B) DNA fragmentation was measured by flow cytometry after 4 days of culture. Data represent islets from 3-25 individual mice per genotype. Statistical significance; *p<0.0001 compared with wild-type islets in 50 mM ribose (one-way ANOVA). (C) DNA fragmentation was measured by flow cytometry after 6 days culture in control medium or medium containing 33.3 mM glucose. Data represent islets from 3-19 individual mice per genotype. Statistical significance; *p<0.0001 compared with wild-type islets in 33.3 mM glucose (one-way ANOVA). (D) Islets from wild-type or bim^{-/-}puma^{-/-} mice were cultured for 4 days in control medium or medium containing 50 mM ribose. DNA fragmentation was measured by flow cytometry. Data represent islets from 3-4 individual mice per genotype. Statistical significance; *p<0.0001 compared with wild-type islets in 50 mM ribose (one-way ANOVA).

FIG. 5: Loss of Bax but not loss of Bak protects islet cells from ribose or glucose induced cytochrome c release and DNA fragmentation. 100 islets from wild-type, bak^{-/-} or bax^{-/-} mice were cultured in control medium or medium containing 50 mM ribose. (A) Cytochrome c release was measured by flow cytometry after 3 days of culture. Data represent islets from 3 individual mice per genotype. Statistical significance; *p<0.0001 compared with wild-type islets in 50 mM ribose (one-way ANOVA). (B) DNA fragmentation was measured by flow cytometry after 4 days of culture. Data represent islets from 6 individual mice per genotype. Statistical significance; *p<0.0001 compared with wild-type islets in 50 mM ribose (one-way ANOVA). (C) DNA fragmentation was measured by flow cytometry after 6 days culture in control medium or medium containing 33.3 mM glucose. Data represent islets from 3 individual mice per genotype. Statistical significance; **p<0.001 compared with wild-type islets in 33.3 mM glucose (one-way ANOVA).

FIG. 6: The initial reduction in insulin secretion caused by exposure to high ribose concentrations is not prevented in Bim-deficient islets. 200 islets from wild-type and Bim-deficient mice were cultured in low glucose or high ribose conditions for 2 days and glucose stimulated insulin secretion assay performed with 20 islets per sample. Data represent islets from 6 individual mice from 3 independent experiments with between 6-18 samples measured per group. Statistical significance; *p<0.01 compared with wild-type unt stimulated with 20 mM glucose, **p<0.001 compared with Bim−/− unt stimulated with 20 mM glucose (two-way ANOVA).

FIG. 7: Ribose induces expression of Puma and Bax in islets. Quantitative RT-PCR of wild-type islets cultured in control medium or medium containing 50 mM ribose, 33.3 mM glucose or 5 μM thapsigargin for 24 or 48 h. Relative RNA expression levels for (A) Puma (B) Bim and (C) Bax were calculated by normalizing to the signal for β-actin in each sample and comparison to islets cultured in control medium. Mean±SEM of 3-4 independent experiments is shown. Statistical significance *p<0.05, **p<0.01 compared with control (two-tailed paired t-test).

FIG. 8: The ER stress pathway contributes to glucose toxicity-induced apoptosis of islet cells. 100 islets from wild type or CHOP^{-/-} mice were cultured for 4 days in control medium or medium containing 50 mM ribose. DNA fragmentation was measured by flow cytometry. Data represent islets from 3-4 individual mice per genotype. Statistical significance; *p<0.0001 compared with wild-type islets in 50 mM ribose (one-way ANOVA).

### Key to the Sequence Listing

SEQ ID NO:1—Amino acid sequence of human Bim, EL isoform (BimEL).

SEQ ID NO:2—Amino acid sequence of human Bim, L isoform (BimL).

SEQ ID NO:3—Amino acid sequence of human Bim, S isoform (BimS).

SEQ ID NO:4—Amino acid sequence of human PUMA, alpha isoform (PUMA alpha).

SEQ ID NO:5—Amino acid sequence of human PUMA, beta isoform (PUMA beta).

SEQ ID NO:6—Amino acid sequence of human PUMA, delta isoform (PUMA delta).

SEQ ID NO:7—Amino acid sequence of human Bax, alpha isoform (Bax alpha).

SEQ ID NO:8—Amino acid sequence of human Bax, beta isoform (Bax beta).

SEQ ID NO:9—Amino acid sequence of human Bax, delta isoform (Bax delta).

SEQ ID NO:10—Amino acid sequence of human Bax, epsilon isoform (Bax epsilon).

SEQ ID NO:11—Amino acid sequence of human Bax, sigma isoform (Bax sigma).

SEQ ID NO:12—Amino acid sequence of human Bax, psi isoform (Bax psi).

SEQ ID NO:13—Amino acid sequence of human Bax, zeta isoform (Bax zeta).

SEQ ID NO:14—mRNA coding sequence for human BimEL isoform.

SEQ ID NO:15—mRNA coding sequence for human BimL isoform.

SEQ ID NO:16—mRNA coding sequence for human BimS isoform.

SEQ ID NO:17—mRNA coding sequence for human PUMA alpha isoform.

SEQ ID NO:18—mRNA coding sequence for human PUMA beta isoform.

SEQ ID NO:19—mRNA coding sequence for human PUMA delta isoform.

SEQ ID NO:20—mRNA coding sequence for human Bax alpha isoform.

SEQ ID NO:21—mRNA coding sequence for human Bax beta isoform.

SEQ ID NO:22—mRNA coding sequence for human Bax delta isoform.

SEQ ID NO:23—mRNA coding sequence for human Bax epsilon isoform.

SEQ ID NO:24—mRNA coding sequence for human Bax sigma isoform.

SEQ ID NO:25—mRNA coding sequence for human Bax psi isoform.

SEQ ID NO:26—mRNA coding sequence for human Bax zeta isoform.

SEQ ID NO:27—Cell penetrating motif.

### DETAILED DESCRIPTION OF THE INVENTION

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

As used herein, the term “disease associated with glucose toxicity” refers to any condition of a subject where abnormal levels of blood glucose have a deleterious effect on the subject. Examples of diseases associated with glucose toxicity include, but are not limited to, Type 2 diabetes, Type 1 diabetes, diabetic neuropathy, diabetic nephropathy or diabetic retinopathy.

As used herein, a “compound” useful for the methods of the invention can be any type of molecule as long as it exerts the desired effect. Examples of suitable types of compounds for use in the invention include, but are not limited to, small carbon based-molecules, polypeptides such as antibodies and polynucleotides such as siRNA, antisense polynuceotides or ribozymes. Examples of compounds suitable for use in the invention are provided herein.

As used herein, the term “Bim polypeptide” refers to a membrane-associated pro-apoptotic Bcl-2 protein family member, which forms heterodimers via its Bcl-2 homology domain 3 (BH3), with all anti-apoptotic Bcl-2 proteins (Bcl-2, Bcl-X_{L}, Bcl-w, Mcl-1 and A1) to promote apoptosis (O'Connor et al., 1998; Willis and Adams, 2005). Bim is also known in the art as Bcl-2-like 11 protein. Several alternatively spliced transcript variants of the Bim gene have been identified giving rise to several isoforms of the Bim protein, including BimEL, BimL and BimS, which vary in apoptotic activity, with BimS being the most potent inducer of cell death (O'Connor et al., 1998; Ewings et al., 2007). Examples of Bim polypeptides include proteins comprising an amino acid sequence provided in SEQ ID NOs 1 to 3, as well as variants and/or mutants thereof.

As used herein, the term “PUMA polypeptide” refers to a pro-apoptotic Bcl-2 protein family member induced by p53 which contains a Bcl-2 homology domain 3 (BH3) and dimerizes with all anti-apoptotic members of the Bcl-2 protein family (Bcl-2, Bcl-X_{L}, Bcl-w, Mcl-1 and A1) via its BH3 domain to promote apoptosis (Nakano and Vousden, 2001; Yu et al., 2001). PUMA is also known in the art as p53 upregulated modulator of apoptosis and Bcl-2-binding component 3 (BBC3). Several alternatively spliced transcript variants of the PUMA gene have been identified giving rise to several isoforms of the PUMA protein, including PUMA alpha, PUMA beta and PUMA delta. Examples of PUMA polypeptides include proteins comprising an amino acid sequence provided in SEQ ID NOs 4 to 6, as well as variants and/or mutants thereof.

As used herein, the term “Bax polypeptide” refers to a pro-apoptotic Bcl-2 protein family member which contains Bcl-2 homology domains 1, 2 and 3 (BH1, BH2 and BH3) and forms heterodimers via these domains with certain anti-apoptotic Bcl-2 family members, including Bcl-2 and Bcl-X_{L}, to promote apoptosis (Gao et al., 2001). Bax is also known in the art as Bcl-2-associated X protein. Several alternatively spliced transcript variants of the Bax gene have been identified giving rise to several isoforms of the Bax protein, including Bax alpha, Bax beta, Bax delta, Bax epsilon, Bax sigma, Bax psi and Bax zeta. Examples of Bax polypeptides include proteins comprising an amino acid sequence provided in SEQ ID NOs 7 to 13, as well as variants and/or mutants thereof.

As used herein, the term “reduces the level of Bim, PUMA and/or Bax activity” refers to a reduction in, or complete eradication of, the biological activity of Bim, PUMA and/or Bax in a cell. In one embodiment, this is achieved by reducing the amount of Bim, PUMA and/or Bax in the cell by limiting the production of one or more of these proteins, and/or increasing the rate of degradation of one or more of these proteins. In another embodiment, the level of Bim, PUMA and/or Bax activity is reduced by limiting the function of one or more of these proteins in the cell, for example wherein the compound is a small molecule which binds Bim, PUMA and/or Bax and limits the ability of the protein(s) to perform its natural apoptosis inducing function.

As used herein, the terms “treating”, “treat” or “treatment” include administering a therapeutically effective amount of a compound(s) described herein sufficient to reduce or eliminate at least one symptom of the specified condition.

As used herein, the terms “preventing”, “prevent” or “prevention” include administering a therapeutically effective amount of a compound(s) described herein sufficient to stop or hinder the development of at least one symptom of the specified condition.

As used herein, the term “subject” refers to any organism capable of having a disease associated with glucose toxicity. In a preferred embodiment, the subject is a mammal. In a particularly preferred embodiment, the subject is a human. Other preferred embodiments include companion animals such as cats and dogs, as well as livestock animals such as horses, cattle, sheep and goats.

By “pancreatic β cell” or “β cell” it is meant a pancreatic islet cell having a phenotype characterized by the expression of markers that normally distinguish the β-cells from the other pancreatic islets cells, such as insulin, Nkx6.1 and/or glucokinase.

As used herein, the term “progenitor cell” refers to a cell capable of differentiation into a cell which is susceptible to glucose toxicity such as, but not limited to, pancreatic β cells, Vasa nervorum cells, proximal tubular epithelial cells, renal glomerulus cells, renal mesangial cells and retinal capillary cells. The term progenitor cell thus encompasses cells that are multipotential, such as stem cells, and cells that are pre-committed to differentiating into cells of a specific lineage.

As used herein, “cells susceptible to exposure to high concentrations of glucose” refers to types of cells that, when in vivo, may be exposed to high glucose concentrations resulting in cell death, particularly in diseased states such as Type 2 diabetes, Type 1 diabetes, diabetic neuropathy, diabetic nephropathy or diabetic retinopathy. Examples of such cells include, but are not limited to, pancreatic β cells or progenitors thereof. In an embodiment, the high concentrations of glucose is at least a concentration of 11 mM of glucose in the blood.

As used herein, “transgenic cells” are cells that have been transformed/transfected with an exogenous polynucleotide, or progeny cells thereof comprising said exogenous polynucleotide.

As used herein, the term “cell culture medium” or variations thereof refers to a medium suitable for the culture, maintenance, proliferation, and/or growth of cells in vitro, especially pancreatic β cells or progenitors thereof. Examples of cell culture media that can be used are disclosed in U.S. Pat. No. 6,670,180 and U.S. Pat. No. 6,730,315. One of skill in the art will recognize that the type of cell culture media useful for the invention can be selected based on the type of cell, tissue, and or organ for which the solution is to be used. For example, where the cells are pancreatic islets, the cell culture medium can be RPMI. Alternative cell culture media, including Eagles Minimal Media, Dulbecco's Modified Eagle's Media, and others known to those with skill in the art, are commercially available (for example, from GIBCO, Long Island, N.Y. and Sigma Chemical Co., St; Louis. Mo.).

As used herein, the term “high sugar concentration” refers to glucose levels (when in vivo, blood glucose levels) that are capable of triggering cell death, for example of pancreatic β cells or progenitors thereof. In an embodiment, the high sugar concentration is at least 11 mM of sugar. In a preferred embodiment, the sugar concentration is determined by measuring monoshaccarides such as glucose and ribose. More preferably, the high sugar concentration is at least 11 mM of glucose.

The present inventors have now shown, for the first time, that Bim, PUMA and Bax are involved in the death of cells in diseases associated with glucose toxicity. This enables compounds that reduce the level of Bim, PUMA and/or Bax activity in a cell to be used in the treatment and/or prevention of diseases associated with glucose toxicity. For example, small molecules which bind Bim, PUMA and/or Bax can be used to reduce the activity of one or more of these proteins. In an embodiment, the small molecule is an organic compound that is not a polymer. In another example, RNA interference can be used to reduce the level of production of Bim, PUMA and/or Bax.

The compound may be, for example, a purified and/or recombinant naturally occurring ligand or a synthetic ligand. The binding between a compound and Bim, PUMA and/or Bax may be mediated by covalent or non-covalent interactions or a combination of covalent and non-covalent interactions. When the interaction of the compound and Bim, PUMA and/or Bax produces a non-covalently bound complex, the binding which occurs is typically electrostatic, hydrogen-bonding, or the result of hydrophilic/lipophilic interactions. In one preferred embodiment, the compound is a purified and/or recombinant polypeptide. Particularly preferred Bim, PUMA and/or Bax binding compounds are purified and/or recombinant anti-Bim, anti-PUMA and/or anti-Bax antibodies or antigenic binding fragments thereof.

In an embodiment, the compound binds specifically to Bim, PUMA and/or Bax. The phrase “specifically binds” means that under particular conditions, the compound binds to Bim, PUMA and/or Bax and does not bind to a significant amount to other, for example, proteins or carbohydrates in the cell. Preferably, the compound specifically binds Bim, PUMA and/or Bax and not other molecules in pancreatic β cells, Vasa nervorum cells, proximal tubular epithelial cells, renal glomerulus cells, renal mesangial cells, retinal capillary cells, or progenitor cells of any one or more thereof. Specific binding under such conditions may require a compound, such an antibody, that is selected for its specificity. In another embodiment, a compound is considered to “specifically binds” to Bim, PUMA and/or Bax if there is a greater than 10-fold difference, and preferably a 25-, 50- or 100-fold greater difference between the binding of the compound to Bim, PUMA and/or Bax when compared to another protein, especially another BH-3 only protein.

In a particularly preferred embodiment, the compound is capable of crossing the membrane of a cell, such as a pancreatic β cell. In some instances this ability can be enhanced or conferred by fusing to the compound a cell-penetrating motif. An example of such a cell-penetrating motif is AAVLLPVLLAAP (SEQ ID NO: 27) described in WO 05/086800. Other examples of cell-penetrating motifs are described in U.S. Pat. No. 5,807,746, U.S. Pat. No. 6,043,339, U.S. Pat. No. 6,495,518, U.S. Pat. No. 6,248,558, U.S. Pat. No. 6,432,680, U.S. Pat. No. 6,780,843, WO 99/49879, WO 01/37821, WO 94/04686 and U.S. Pat. No. 6,316,003.

In an embodiment, the compound which reduces Bax activity is a substituted amine derivative described in US 20030216427.

In yet a further embodiment, molecules described in US 2006/018687 and WO 2008/040087 can also be used to reduce Bim activity in a cell.

In one embodiment, a compound useful for the methods of the invention is a polypeptide. Examples include antibodies or antigenic binding fragments thereof, as well as fragments of proteins which naturally bind Bim, PUMA and/or Bax in a cell which act as antagonists of Bim, PUMA and/or Bax activity.

The terms “polypeptide” and “protein” are generally used interchangeably and refer to a single polypeptide chain which may or may not be modified by addition of non-amino acid groups. It would be understood that such polypeptide chains may associate with other polypeptides or proteins or other molecules such as co-factors. The terms “proteins” and “polypeptides” as used herein also include variants, mutants, biologically active fragments, modifications, analogues and/or derivatives of the polypeptides described herein.

The term “recombinant” in the context of a protein refers to the polypeptide when produced by a cell, or in a cell-free expression system, in an altered amount or at an altered rate compared to its native state. In one embodiment the cell is a cell that does not naturally produce the polypeptide. However, the cell may be a cell which comprises a non-endogenous gene that causes an altered, preferably increased, amount of the polypeptide to be produced. A recombinant polypeptide of the invention includes polypeptides which have not been separated from other components of the transgenic (recombinant) cell, or cell-free expression system, in which it is produced, and polypeptides produced in such cells or cell-free systems which are subsequently purified away from at least some other components.

In an embodiment, the compound which reduces Bax activity is Bax inhibitor protein-1, Bax inhibitor protein-2 or related molecules described in U.S. Pat. No. 5,837,838.

In another embodiment, the compound which reduces Bax activity is a RY domain peptide or related molecules described in U.S. Pat. No. 6,849,603.

In another embodiment, the compound which reduces Bax activity is a fragment of Bax, Bcl-2 or Bad, such as a 20 amino acid Bax BH3 peptide, as described by Shangary and Johnson (2002).

If desired, unnatural amino acids or chemical amino acid analogues can be introduced as a substitution or addition into a polypeptide. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Ca-methyl amino acids, Nα-methyl amino acids, and amino acid analogues in general.

Also included within the scope of the invention is the use of polypeptides which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide.

Polypeptides can be produced in a variety of ways, including production and recovery of natural polypeptides, production and recovery of recombinant polypeptides, and chemical synthesis of the polypeptides. In one embodiment, an isolated polypeptide of the present invention is produced by culturing a cell capable of expressing the polypeptide under conditions effective to produce the polypeptide, and recovering the polypeptide. A preferred cell to culture is a recombinant cell of the present invention. Effective culture conditions include, but are not limited to, effective media, bioreactor, temperature, pH and oxygen conditions that permit polypeptide production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, tissue culture flasks, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Antibodies may exist as intact immunoglobulins, or as modifications in a variety of forms including, for example, but not limited to, domain antibodies including either the variable heavy (V_{H}) chain or variable heavy (V_{L}) chain, a dimer of the heavy chain variable region (VHH, as described for a camelid), a dimer of the light chain variable region (VLL), Fv fragments containing only the light and heavy chain variable regions, or Fd fragments containing the heavy chain variable region and the CH1 domain. A scFv consisting of the variable regions of the heavy and light chains linked together to form a single-chain antibody (Bird et al., 1988; Huston et al., 1988) and oligomers of scFvs such as diabodies and triabodies are also encompassed by the term “antibody”. Also encompassed are fragments of antibodies such as Fab, (Fab′)₂ and FabFc₂ fragments which contain the variable regions and parts of the constant regions. CDR-grafted antibody fragments and oligomers of antibody fragments are also encompassed. The heavy and light chain components of an Fv may be derived from the same antibody or different antibodies thereby producing a chimeric Fv region. The antibody may be of animal (for example mouse, rabbit or rat) or human origin or may be chimeric (Morrison et al., 1984) or humanized (Jones et al., 1986).

The antibodies may be Fv regions comprising a V_{L} and a V_{H}. The light and heavy chains may be joined directly or through a linker. As used herein a linker refers to a molecule that is covalently linked to the light and heavy chain and provides enough spacing and flexibility between the two chains such that they are able to achieve a conformation in which they are capable of specifically binding the epitope to which they are directed. Protein linkers are particularly preferred as they may be expressed as an intrinsic component of the Ig portion of the fusion polypeptide.

In a preferred embodiment, the antibody or fragment thereof used in the methods of the invention is an internalizing antibody. An “internalizing antibody” is an antibody that is capable of being transported into a cell. Methods for producing and/or selecting internalizing antibodies are known in the art, such as those methods described in Poul et al. (2000) and Becerril et al. (1999).

A variety of immuno-assay formats may be used to select antibodies specifically immunoreactive with Bim, PUMA and/or Bax. For example, surface labelling and flow cytometric analysis or solid-phase ELISA immunoassays are routinely used to select antibodies specifically immunoreactive with a protein or carbohydrate. See Harlow & Lane (supra) for a description of immuno-assay formats and conditions that can be used to determine specific immunoreactivity. In another embodiment, recombinantly produced single chain scFv antibody, preferably a humanized scFv, is used in the methods of the invention.

Examples of antibodies which bind and reduce Bax activity are described in WO 2007/025388 and U.S. Pat. No. 6,245,885.

Examples of antibodies which bind and reduce PUMA activity are described in WO 00/26228.

An example of an antibody which binds and reduces Bim activity is 3C5 described by O'Reilly et al. (2009).

Anti-Bim, anti-PUMA and anti-Bax antibodies can be obtained from commercial sources such as Alexis, Stressgen or Cell Signaling Technology (Danvers, Mass., USA).

Monoclonal antibodies directed against Bim, PUMA and/or Bax epitopes can be readily produced by one skilled in the art.

The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against Bim, PUMA and/or Bax epitopes can be screened for various properties; i.e. for isotype and epitope affinity.

Animal-derived monoclonal antibodies can be used for both direct in vivo and extracorporeal immunotherapy. However, it has been observed that when, for example, mouse-derived monoclonal antibodies are used in humans as therapeutic compounds, the patient produces human anti-mouse antibodies. Thus, animal-derived monoclonal antibodies are not preferred for therapy, especially for long term use. With established genetic engineering techniques it is possible, however, to create chimeric or humanized antibodies that have animal-derived and human-derived portions. The animal can be, for example, a mouse or other rodent such as a rat.

If the variable region of the chimeric antibody is, for example, mouse-derived while the constant region is human-derived, the chimeric antibody will generally be less immunogenic than a “pure” mouse-derived monoclonal antibody. These chimeric antibodies would likely be more suited for therapeutic use, should it turn out that “pure” mouse-derived antibodies are unsuitable.

Methodologies for generating chimeric antibodies are available to those in the art. For example, the light and heavy chains can be expressed separately, using, for example, immunoglobulin light chain and immunoglobulin heavy chains in separate plasmids. These can then be purified and assembled in vitro into complete antibodies; methodologies for accomplishing such assembly have been described (see, for example, Sun et al., 1986). Such a DNA construct may comprise DNA encoding functionally rearranged genes for the variable region of a light or heavy chain of an anti-Bim, anti-PUMA or anti-Bax antibody linked to DNA encoding a human constant region. Lymphoid cells such as myelomas or hybridomas transfected with the DNA constructs for light and heavy chain can express and assemble the antibody chains.

In vitro reaction parameters for the formation of IgG antibodies from reduced isolated light and heavy chains have also been described. Co-expression of light and heavy chains in the same cells to achieve intracellular association and linkage of heavy and light chains into complete H2L2 IgG antibodies is also possible. Such co-expression can be accomplished using either the same or different plasmids in the same host cell.

In another preferred embodiment of the present invention the antibody is humanized, that is, an antibody produced by molecular modeling techniques wherein the human content of the antibody is maximised while causing little or no loss of binding affinity attributable to the variable region of, for example, a parental rat, rabbit or murine antibody.

The methods described below are applicable to the humanisation of anti-Bim, anti-PUMA and/or anti-Bax antibodies antibodies.

There are several factors to consider in deciding which human antibody sequence to use during the humanisation. The humanisation of light and heavy chains are considered independently of one another, but the reasoning is basically similar for each.

This selection process is based on the following rationale: A given antibody's antigen specificity and affinity is primarily determined by the amino acid sequence of the variable region CDRs. Variable domain framework residues have little or no direct contribution. The primary function of the framework regions is to hold the CDRs in their proper spatial orientation to recognize antigen. Thus the substitution of animal, for example, rodent CDRs into a human variable domain framework is most likely to result in retention of their correct spatial orientation if the human variable domain framework is highly homologous to the animal variable domain from which they originated. A human variable domain should preferably be chosen therefore that is highly homologous to the animal variable domain(s). A suitable human antibody variable domain sequence can be selected as follows.

Step 1. Using a computer program, search all available protein (and DNA) databases for those human antibody variable domain sequences that are most homologous to the animal-derived antibody variable domains. The output of a suitable program is a list of sequences most homologous to the animal-derived antibody, the percent homology to each sequence, and an alignment of each sequence to the animal-derived sequence. This is done independently for both the heavy and light chain variable domain sequences. The above analyses are more easily accomplished if only human immunoglobulin sequences are included.

Step 2. List the human antibody variable domain sequences and compare for homology. Primarily the comparison is performed on length of CDRs, except CDR3 of the heavy chain which is quite variable. Human heavy chains and Kappa and Lambda light chains are divided into subgroups; Heavy chain 3 subgroups, Kappa chain 4 subgroups, Lambda chain 6 subgroups. The CDR sizes within each subgroup are similar but vary between subgroups. It is usually possible to match an animal-derived antibody CDR to one of the human subgroups as a first approximation of homology. Antibodies bearing CDRs of similar length are then compared for amino acid sequence homology, especially within the CDRs, but also in the surrounding framework regions. The human variable domain which is most homologous is chosen as the framework for humanisation.

An antibody may be humanized by grafting the desired CDRs onto a human framework according to EP-A-0239400. A DNA sequence encoding the desired reshaped antibody can therefore be made beginning with the human DNA whose CDRs it is wished to reshape. The animal-derived variable domain amino acid sequence containing the desired CDRs is compared to that of the chosen human antibody variable domain sequence. The residues in the human variable domain are marked that need to be changed to the corresponding residue in the animal to make the human variable region incorporate the animal-derived CDRs. There may also be residues that need substituting in, adding to or deleting from the human sequence.

Oligonucleotides are synthesized that can be used to mutagenize the human variable domain framework to contain the desired residues. Those oligonucleotides can be of any convenient size. One is normally only limited in length by the capabilities of the particular synthesizer one has available. The method of oligonucleotide-directed in vitro mutagenesis is well known.

Alternatively, humanisation may be achieved using the recombinant polymerase chain reaction (PCR) methodology of WO 92/07075. Using this methodology, a CDR may be spliced between the framework regions of a human antibody. In general, the technique of WO 92/07075 can be performed using a template comprising two human framework regions, AB and CD, and between them, the CDR which is to be replaced by a donor CDR. Primers A and B are used to amplify the framework region AB, and primers C and D used to amplify the framework region CD. However, the primers B and C each also contain, at their 5′ ends, an additional sequence corresponding to all or at least part of the donor CDR sequence. Primers B and C overlap by a length sufficient to permit annealing of their 5′ ends to each other under conditions which allow a PCR to be performed. Thus, the amplified regions AB and CD may undergo gene splicing by overlap extension to produce the humanized product in a single reaction.

Following the mutagenesis reactions to reshape the antibody, the mutagenised DNAs can be linked to an appropriate DNA encoding a light or heavy chain constant region, cloned into an expression vector, and transfected into host cells, preferably mammalian cells. These steps can be carried out in routine fashion. A reshaped antibody may therefore be prepared by a process comprising:
(a) preparing a first replicable expression vector including a suitable promoter operably linked to a DNA sequence which encodes at least a variable domain of an Ig heavy or light chain, the variable domain comprising framework regions from a human antibody and the CDRs required for the humanized antibody of the invention;
(b) preparing a second replicable expression vector including a suitable promoter operably linked to a DNA sequence which encodes at least the variable domain of a complementary Ig light or heavy chain respectively;
(c) transforming a cell line with the first or both prepared vectors; and
(d) culturing said transformed cell line to produce said altered antibody.

Preferably the DNA sequence in step (a) encodes both the variable domain and each constant domain of the human antibody chain. The humanized antibody can be prepared using any suitable recombinant expression system. The cell line which is transformed to produce the altered antibody may be a CHO cell line or an immortalised mammalian cell line, which is advantageously of lymphoid origin, such as a myeloma, hybridoma, trioma or quadroma cell line. The cell line may also comprise a normal lymphoid cell, such as a β-cell, which has been immortalised by transformation with a virus, such as the Epstein-Barr virus. Most preferably, the immortalised cell line is a myeloma cell line or a derivative thereof.

The CHO cells used for expression of the antibodies may be dihydrofolate reductase (dhfr) deficient and so dependent on thymidine and hypoxanthine for growth. The parental dhfr⁻ CHO cell line is transfected with the DNA encoding the antibody and dhfr gene which enables selection of CHO cell transformants of dhfr positive phenotype. Selection is carried out by culturing the colonies on media devoid of thymidine and hypoxanthine, the absence of which prevents untransformed cells from growing and transformed cells from resalvaging the folate pathway and thus bypassing the selection system. These transformants usually express low levels of the DNA of interest by virtue of co-integration of transfected DNA of interest and DNA encoding dhfr. The expression levels of the DNA encoding the antibody may be increased by amplification using methotrexate (MTX). This drug is a direct inhibitor of the enzyme dhfr and allows isolation of resistant colonies which amplify their dhfr gene copy number sufficiently to survive under these conditions. Since the DNA sequences encoding dhfr and the antibody are closely linked in the original transformants, there is usually concomitant amplification, and therefore increased expression of the desired antibody.

Another preferred expression system for use with CHO or myeloma cells is the glutamine synthetase (GS) amplification system described in WO 87/04462. This system involves the transfection of a cell with DNA encoding the enzyme GS and with DNA encoding the desired antibody. Cells are then selected which grow in glutamine free medium and can thus be assumed to have integrated the DNA encoding GS. These selected clones are then subjected to inhibition of the enzyme GS using methionine sulphoximine (Msx). The cells, in order to survive, will amplify the DNA encoding GS with concomitant amplification of the DNA encoding the antibody.

Although the cell line used to produce the humanized antibody is preferably a mammalian cell line, any other suitable cell line, such as a bacterial cell line or a yeast cell line, may alternatively be used. In particular, it is envisaged that E. coli-derived bacterial strains could be used. The antibody obtained is checked for functionality. If functionality is lost, it is necessary to return to step (2) and alter the framework of the antibody.

Once expressed, the whole antibodies, their dimers, individual light and heavy chains, or other immunoglobulin forms can be recovered and purified according to standard procedures of the art, including ammonium sulfate precipitation, affinity columns, column chromatography, gel electrophoresis and the like (see, generally, Scopes, R., Protein Purification, Springer-Verlag, N.Y. (1982)). Substantially pure immunoglobulins of at least about 90 to 95% homogeneity are preferred, and 98 to 99% or more homogeneity most preferred, for pharmaceutical uses. Once purified, partially or to homogeneity as desired, a humanized antibody may then be used therapeutically or in developing and performing assay procedures, immunofluorescent stainings, and the like (see, generally, Lefkovits and Pernis (editors), Immunological Methods, Vols. I and II, Academic Press, (1979 and 1981)).

Antibodies with fully human variable regions against Bim, PUMA and/or Bax can also be prepared by administering the antigen to a transgenic animal which has been modified to produce such antibodies in response to antigenic challenge, but whose endogenous loci have been disabled. Various subsequent manipulations can be performed to obtain either antibodies per se or analogs thereof (see, for example, U.S. Pat. No. 6,075,181).

Genes encoding antibodies, both light and heavy chain genes or portions thereof, e.g., single chain Fv regions, may be cloned from a hybridoma cell line. They may all be cloned using the same general strategy. Typically, for example, poly(A)⁺mRNA extracted from the hybridoma cells is reverse transcribed using random hexamers as primers. For Fv regions, the V_{H} and V_{L} domains are amplified separately by two polymerase chain reactions (PCR). Heavy chain sequences may be amplified using 5′ end primers which are designed according to the amino-terminal protein sequences of the anti-Bim, anti-PUMA or anti-Bax antibodies heavy chains respectively and 3′ end primers according to consensus immunoglobulin constant region sequences (Kabat et al., Sequences of Proteins of Immunological Interest. 5th edition. U.S. Department of Health and Human Services, Public Health Service, National Institutes of Health, Bethesda, Md. (1991)). Light chain Fv regions are amplified using 5′ end primers designed according to the amino-terminal protein sequences of anti-Bim, anti-PUMA or anti-Bax antibodies light chains and in combination with the primer C-kappa. One of skill in the art would recognize that many suitable primers may be employed to obtain Fv regions.

The PCR products are subcloned into a suitable cloning vector. Clones containing the correct size insert by DNA restriction are identified. The nucleotide sequence of the heavy or light chain coding regions may then be determined from double-stranded plasmid DNA using sequencing primers adjacent to the cloning site. Commercially available kits (e.g., the Sequenase™ kit, United States Biochemical Corp., Cleveland, Ohio, USA) may be used to facilitate sequencing the DNA. DNA encoding the Fv regions may be prepared by any suitable method, including, for example, amplification techniques such as PCR and LCR.

Chemical synthesis produces a single-stranded oligonucleotide. This may be converted into double-stranded DNA by hybridization with a complementary sequence, or by polymerization with a DNA polymerase using the single strand as a template. While it is possible to chemically synthesize an entire single chain Fv region, it is preferable to synthesize a number of shorter sequences (about 100 to 150 bases) that are later ligated together.

Alternatively, sub-sequences may be cloned and the appropriate subsequences cleaved using appropriate restriction enzymes. The fragments may then be ligated to produce the desired DNA sequence.

Once the Fv variable light and heavy chain DNA is obtained, the sequences may be ligated together, either directly or through a DNA sequence encoding a peptide linker, using techniques well known to those of skill in the art. In one embodiment, heavy and light chain regions are connected by a flexible peptide linker (e.g., (Gly₄Ser)₃) which starts at the carboxyl end of the heavy chain Fv domain and ends at the amino terminus of the light chain Fv domain. The entire sequence encodes the Fv domain in the form of a single-chain antigen binding protein.

In some embodiments, a polypeptide can be delivered/administered by providing a polynucleotide encoding the polypeptide. In other embodiments, the polynucleotide per se can be a useful compound for use in the invention (for example, using RNAi).

The term “polynucleotide” is used interchangeably herein with the term “nucleic acid”.

The term “exogenous” in the context of a polynucleotide refers to the polynucleotide when present in a cell, or in a cell-free expression system, in an altered amount compared to its native state. In one embodiment, the cell is a cell that does not naturally comprise the polynucleotide. However, the cell may be a cell which comprises a non-endogenous polynucleotide resulting in an altered, preferably increased, amount of production of the encoded polypeptide.

Usually, monomers of a polynucleotide or oligonucleotide are linked by phosphodiester bonds or analogs thereof to form oligonucleotides ranging in size from relatively short monomeric units, e.g., 19-23, to several hundreds of monomeric units. Analogs of phosphodiester linkages include: phosphorothioate, phosphorodithioate, phosphoroselenoate, phosphorodiselenoate, phosphoroanilothioate, phosphoranilidate and phosphoramidate.

The term “antisense polynucleotide” shall be taken to mean a DNA or RNA, or combination thereof, molecule that is complementary to at least a portion of a specific mRNA molecule encoding a polypeptide of the invention and capable of interfering with a post-transcriptional event such as mRNA translation. The use of antisense methods is well known in the art (see for example, G. Hartmann and S. Endres, Manual of Antisense Methodology, Kluwer (1999)).

An antisense polynucleotide for use in the methods of the invention will hybridize to a target polynucleotide under physiological conditions. As used herein, the term “an antisense polynucleotide which hybridises under physiological conditions” means that the polynucleotide (which is fully or partially single-stranded) is at least capable of forming a double-stranded polynucleotide with mRNA encoding a protein, such as those provided in any one of SEQ ID NOs 14 to 26 under normal conditions in a cell, preferably a human β cell.

Antisense molecules may include sequences that correspond to the structural genes or for sequences that effect control over the gene expression or splicing event. For example, the antisense sequence may correspond to the targeted coding region of the genes of the invention, or the 5′-untranslated region (UTR) or the 3′-UTR or a combination of these. It may be complementary in part to intron sequences, which may be spliced out during or after transcription, preferably only to exon sequences of the target gene. In view of the generally greater divergence of the UTRs, targeting these regions provides greater specificity of gene inhibition.

The length of the antisense sequence should be at least 19 contiguous nucleotides, preferably at least 50 nucleotides, and more preferably at least 100, 200, 500 or 1000 nucleotides. The full-length sequence complementary to the entire gene transcript may be used. The length is most preferably 100-2000 nucleotides. The degree of identity of the antisense sequence to the targeted transcript should be at least 90% and more preferably 95-100%. The antisense RNA molecule may of course comprise unrelated sequences which may function to stabilize the molecule.

The term catalytic polynucleotide/nucleic acid refers to a DNA molecule or DNA-containing molecule (also known in the art as a “deoxyribozyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are particularly useful in this invention are the hammerhead ribozyme (Haseloff and Gerlach, 1988; Perriman et al., 1992) and the hairpin ribozyme (Shippy et al., 1999).

The ribozymes for use in the methods of this invention and DNA encoding the ribozymes can be chemically synthesized using methods well known in the art. The ribozymes can also be prepared from a DNA molecule (that upon transcription, yields an RNA molecule) operably linked to an RNA polymerase promoter, e.g., the promoter for T7 RNA polymerase or SP6 RNA polymerase. Accordingly, also provided by this invention is a nucleic acid molecule, i.e., DNA or cDNA, coding for a catalytic polynucleotide of the invention. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette. After synthesis, the RNA molecule can be modified by ligation to a DNA molecule having the ability to stabilize the ribozyme and make it resistant to RNase.

As with antisense polynucleotides described herein, catalytic polynucleotides for use in the methods of the invention should also be capable of hybridizing to a target nucleic acid molecule (for example an mRNA encoding any polypeptide provided in SEQ ID NOs 1 to 13) under “physiological conditions”, namely those conditions within a cell (especially conditions in an animal cell such as a human cell).

The terms “RNA interference”, “RNAi” or “gene silencing” refers generally to a process in which a double-stranded RNA molecule reduces the expression of a nucleic acid sequence with which the double-stranded RNA molecule shares substantial or total homology. However, it has more recently been shown that RNA interference can be achieved using non-RNA double-stranded molecules (see, for example, US 20070004667).

RNA interference (RNAi) is particularly useful for specifically inhibiting the production of a particular RNA and/or protein. Although not wishing to be limited by theory, Waterhouse et al. (1998) have provided a model for the mechanism by which dsRNA (duplex RNA) can be used to reduce protein production. This technology relies on the presence of dsRNA molecules that contain a sequence that is essentially identical to the mRNA of the gene of interest or part thereof, in this case an mRNA encoding a polypeptide according to the invention. Conveniently, the dsRNA can be produced from a single promoter in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for the present invention is well within the capacity of a person skilled in the art, particularly considering Waterhouse et al. (1998), Smith et al. (2000), WO 99/32619, WO 99/53050, WO 99/49029, and WO 01/34815.

The present invention includes the use of nucleic acid molecules comprising and/or encoding double-stranded regions for RNA interference. The nucleic acid molecules are typically RNA but may comprise chemically-modified nucleotides and non-nucleotides.

The double-stranded regions should be at least 19 contiguous nucleotides, for example about 19 to 23 nucleotides, or may be longer, for example 30 or 50 nucleotides, or 100 nucleotides or more. The full-length sequence corresponding to the entire gene transcript may be used. Preferably, they are about 19 to about 23 nucleotides in length.

The degree of identity of a double-stranded region of a nucleic acid molecule to the targeted transcript should be at least 90% and more preferably 95-100%. The nucleic acid molecule may of course comprise unrelated sequences which may function to stabilize the molecule.

The term “short interfering RNA” or “siRNA” as used herein refers to a nucleic acid molecule which comprises ribonucleotides capable of inhibiting or down regulating gene expression, for example by mediating RNAi in a sequence-specific manner, wherein the double-stranded portion is less than 50 nucleotides in length, preferably about 19 to about 23 nucleotides in length. For example the siRNA can be a nucleic acid molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary.

As used herein, the term siRNA is meant to be equivalent to other terms used to describe nucleic acid molecules that are capable of mediating sequence specific RNAi, for example micro-RNA (miRNA), short hairpin RNA (shRNA), short interfering oligonucleotide, short interfering nucleic acid (siNA), short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siRNA molecules of the invention can be used to epigenetically silence genes at both the post-transcriptional level or the pre-transcriptional level. In a non-limiting example, epigenetic regulation of gene expression by siRNA molecules of the invention can result from siRNA mediated modification of chromatin structure to alter gene expression.

Preferred small interfering RNA (“siRNA”) molecules comprise a nucleotide sequence that is identical to about 19 to 23 contiguous nucleotides of the target mRNA. In an embodiment, the target mRNA sequence commences with the dinucleotide AA, comprises a GC-content of about 30-70% (preferably, 30-60%, more preferably 40-60% and more preferably about 45%-55%), and does not have a high percentage identity to any nucleotide sequence other than the target in the genome of the subject in which it is to be introduced, e.g., as determined by standard BLAST search.

By “shRNA” or “short-hairpin RNA” is meant an siRNA molecule where less than about 50 nucleotides, preferably about 19 to about 23 nucleotides, is base paired with a complementary sequence located on the same RNA molecule, and where said sequence and complementary sequence are separated by an unpaired region of at least about 4 to 15 nucleotides which forms a single-stranded loop above the stem structure created by the two regions of base complementarity. An example of a sequence of a single-stranded loop is 5′ UUCAAGAGA 3′.

Included shRNAs are dual or bi-finger and multi-finger hairpin dsRNAs, in which the RNA molecule comprises two or more of such stem-loop structures separated by single-stranded spacer regions.

There are well-established criteria for designing siRNAs (see, for example, Elbashire et al., 2001; Amarzguioui et al., 2004; Reynolds et al., 2004). Details can be found in the websites of several commercial vendors such as Ambion, Dharmacon, GenScript, and OligoEngine. Typically, a number of siRNAs have to be generated and screened in order to compare their effectiveness.

Once designed, the dsRNAs for use in the method of the present invention can be generated by any method known in the art, for example, by in vitro transcription, recombinantly, or by synthetic means. siRNAs can be generated in vitro by using a recombinant enzyme, such as T7 RNA polymerase, and DNA oligonucleotide templates, or can be prepared in vivo, for example, in cultured cells.

In addition, strategies have been described for producing a hairpin siRNA from vectors containing, for example, a RNA polymerase III promoter. Various vectors have been constructed for generating hairpin siRNAs in host cells using either an H1-RNA or an snU6 RNA promoter. A RNA molecule as described above (e.g., a first portion, a linking sequence, and a second portion) can be operably linked to such a promoter. When transcribed by RNA polymerase III, the first and second portions form a duplexed stem of a hairpin and the linking sequence forms a loop. The pSuper vector (OligoEngines Ltd., Seattle, Wash.) also can be used to generate siRNA.

Modifications or analogs of nucleotides can be introduced to improve the properties of the nucleic acid molecules of the invention. Improved properties include increased nuclease resistance and/or increased ability to permeate cell membranes. Accordingly, the terms “nucleic acid molecule” and “double-stranded RNA molecule” includes synthetically modified bases such as, but not limited to, inosine, xanthine, hypoxanthine, 2-aminoadenine, 6-methyl-, 2-propyl- and other alkyl-adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

Examples of RNAi molecules that can be used to reduce Bax activity are described in US 20080069840.

Examples of RNAi molecules that can be used to reduce Bim activity are described in Gong et al. (2007), Bouillet et al. (2005), Essafi et al. (2005), Kuroda et al. (2006) and US 20080025958.

Examples of RNAi molecules that can be used to reduce PUMA activity are described in US 20080025958 and Hemann et al. (2004).

MicroRNA regulation is a clearly specialized branch of the RNA silencing pathway that evolved towards gene regulation, diverging from conventional RNAi/PTGS. MicroRNAs are a specific class of small RNAs that are encoded in gene-like elements organized in a characteristic inverted repeat. When transcribed, microRNA genes give rise to stem-looped precursor RNAs from which the microRNAs are subsequently processed. MicroRNAs are typically about 21 nucleotides in length. The released miRNAs are incorporated into RISC-like complexes containing a particular subset of Argonaute proteins that exert sequence-specific gene repression (see, for example, Millar and Waterhouse, 2005; Pasquinelli et al., 2005; Almeida and Allshire, 2005).

Polynucleotides useful for the invention are typically inserted into a recombinant vector. Such a vector contains exogenous polynucleotide sequences, that is polynucleotide sequences that are not naturally found adjacent to polynucleotide molecules useful for the present invention and that preferably are derived from a species other than the species from which the polynucleotide molecule(s) are derived. The vector can be either RNA or DNA, either prokaryotic or eukaryotic, and typically is a transposon (such as described in U.S. Pat. No. 5,792,294), a virus or a plasmid.

A particularly preferred recombinant vector comprises the polynucleotide(s) operably linked to an expression vector. The phrase operably linked refers to insertion of a polynucleotide molecule into an expression vector in a manner such that the molecule is able to be expressed when transformed into a host cell. As used herein, an expression vector is a DNA or RNA vector that is capable of transforming a host cell and of effecting expression of a specified polynucleotide molecule. Preferably, the expression vector is also capable of replicating within the host cell. Expression vectors can be either prokaryotic or eukaryotic, and are typically viruses or plasmids. Expression vectors include any vectors that function (i.e., direct gene expression) in recombinant cells, including in bacterial, fungal, endoparasite, arthropod, animal and plant cells, most preferably animal cells. Vectors of the invention can also be used to produce the polypeptide in a cell-free expression system, with such systems are well known in the art.

“Operably linked” as used herein refers to a functional relationship between two or more nucleic acid (e.g., DNA) segments. Typically, it refers to the functional relationship of transcriptional regulatory element to a transcribed sequence. For example, a promoter is operably linked to a coding sequence, such as a polynucleotide defined herein, if it stimulates or modulates the transcription of the coding sequence in an appropriate host cell and/or in a cell-free expression system. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

In particular, expression vectors of the present invention contain regulatory sequences such as transcription control sequences, translation control sequences, origins of replication, and other regulatory sequences that are compatible with the recombinant (host) cell and that control the expression of polynucleotide molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in animal cells.

In one embodiment, the construct contains a promoter to facilitate expression of the polynucleotide within a pancreatic β cell. The promoter may be a strong, viral promoter that functions in eukaryotic cells such as a promoter from cytomegalovirus (CMV), mouse mammary tumor virus (MMTV), Rous sarcoma virus (RSV), or adenovirus. More specifically, exemplary promoters include the promoter from the immediate early gene of human CMV and the promoter from the long terminal repeat (LTR) of RSV.

Alternatively, the promoter used may be a strong general eukaryotic promoter such as the actin gene promoter. In one embodiment, the promoter used may be a tissue-specific promoter. For example, the promoter used in the construct may be a pancreas specific promoter, a duct cell specific promoter or a stem cell specific promoter.

In another embodiment, the promoter is a regulated promoter, such as a tetracycline-regulated promoter, expression from which can be regulated by exposure to an exogenous substance (e.g., tetracycline.).

Other components such as a marker (e.g., an antibiotic resistance gene (such as an ampicillin resistance gene) or β-galactosidase) aid in selection or identification of cells containing and/or expressing the construct, an origin of replication for stable replication of the construct in a bacterial cell (preferably, a high copy number origin of replication), a nuclear localization signal, or other elements which facilitate production of the construct, the protein encoded thereby, or both.

A compound useful for the methods of the invention can be introduced into a cell in vitro, for example a pancreatic β cell, a Vasa nervorum cell, a proximal tubular epithelial cell, a renal glomerulus cell, a renal mesangial cell, a retinal capillary cell, or progenitor cell of any one or more thereof. In a preferred embodiment, the compound delivered to a cell in vitro is a polynucleotide. The cell can then be introduced, possibly following culturing for one or more cell divisions, into the subject.

Primary and secondary cells to be genetically engineered can be obtained from a variety of tissues and can include cell types that can be maintained and propagated in culture. Primary cells are preferably obtained from the individual to whom the genetically engineered primary or secondary cells will be administered. However, primary cells may be obtained from a donor (i.e., an individual other than the recipient).

The term “primary cell” includes cells present in a suspension of cells isolated from a tissue source (prior to their being plated, i.e., attached to a tissue culture substrate such as a dish or flask), cells present in an explant derived from tissue, both of the previous types of cells plated for the first time, and cell suspensions derived from these plated cells. The term “secondary cell” or “cell strain” refers to cells at all subsequent steps in culturing. Secondary cells are cell strains which consist of primary cells which have been passaged one or more times.

Cells can be obtained by standard methods such a punch biopsy or other surgical methods of obtaining a tissue source of the primary cell type of interest. For example, a biopsy can be used to obtain pancreatic tissue, as a source of pancreatic β cells. A mixture of primary cells can be obtained from the tissue, using known methods, such as enzymatic digestion or explanting. If enzymatic digestion is used, enzymes such as collagenase, hyaluronidase, dispase, pronase, trypsin, elastase and chymotrypsin can be used.

The resulting primary cell mixture can be transfected directly, or it can be cultured first, removed from the culture plate and resuspended before transfection is carried out. Primary cells or secondary cells are combined with exogenous nucleic acid sequence to, e.g., stably integrate into their genomes, and treated in order to accomplish transfection. As used herein, the term “transfection” includes a variety of techniques for introducing an exogenous nucleic acid into a cell including calcium phosphate or calcium chloride precipitation, microinjection, DEAE-dextrin-mediated transfection, lipofection or electroporation, all of which are routine in the art.

Examples of transplanting cells, such as genetically engineered cells, into the pancreas are provided in Docherty (1997), Hegre et al. (1976), Sandler et al. (1997), Calafiore (1997), Kenyon et al. (1996), Chick et al. (1977), Bonner-Weir et al. (2000), US 20020081285 and US 20020177228. In general, the cells can be implanted into the pancreas, or to any practical or convenient site, e.g., subcutaneous site, liver, peritoneum.

Transfected primary or secondary cells undergo sufficient numbers of doubling to produce either a clonal cell strain or a heterogeneous cell strain of sufficient size to provide the therapeutic protein or polynucleotide to an individual in effective amounts. The number of required cells in a transfected clonal heterogeneous cell strain is variable and depends on a variety of factors, including but not limited to, the use of the transfected cells, the functional level of the exogenous DNA in the transfected cells, the site of implantation of the transfected cells (for example, the number of cells that can be used is limited by the anatomical site of implantation), and the age, surface area, and clinical condition of the patient.

The transfected cells, e.g., cells produced as described herein, can be introduced into an individual to whom the compound is to be delivered. Various routes of administration and various sites (e.g., renal sub capsular, subcutaneous, central nervous system (including intrathecal), intravascular, intrahepatic, intrasplanchnic, intraperitoneal (including intraomental), intramuscularly) can be used. Once implanted in an individual, the transfected cells produce the product encoded by the exogenous polynucleotide or are affected by the heterologous polynucleotide itself. For example, an individual who suffers from a diabetes-related disorder (e.g., type 2 diabetes, type 1 diabetes, impaired glucose tolerance, insulin resistance, or beta cell dysfunction) is a candidate for implantation of cells producing an compound described herein.

The cells may be transplanted or infused alone or in association with a pharmaceutically acceptable carrier or medium. The present invention contemplates the use of the carrier or medium to introduce other compounds, such as immuno-suppressive compounds, therapeutic compounds mitogenic compounds or differentiating agents, into the patient in conjunction with the cells.

Alternatively, the cells can be embedded in a biocompatible medium such as an extracellular matrix that will promote survival and/or proliferation and differentiation of these cells in vivo. The matrix can function as “scaffolding” that holds the cells in place. Or the cells can be administered in a biocompatible medium becomes a semi-solid or solid matrix in situ. Such extracellular matrices are known in the art and may be a natural matrix or may be a matrix that is based on natural polymers, such as collagen and its derivatives, fibronectin, polylactic acid or polyglycolic acid. The present invention also contemplates the incorporation of other therapeutically useful compounds into the matrix with the cells such that the cells and the compound can be delivered concomitantly to, for example, the pancreas, such as a compound which exerts a therapeutic effect in the mammal or that produces a biologically active molecule that has a growth or trophic effect on the transplanted cells, or that induces differentiation of the cells into a particular phenotypic lineage.

Therapeutic polynucleotide molecules described herein may be employed in accordance with the present invention by expression of such polynucleotides in treatment modalities often referred to as “gene therapy”. The invention includes the use of targeted expression vectors for in vivo transfection and expression of the polynucleotide described herein, in particular cell types, especially pancreatic β-cells. Expression constructs of such components can be administered in any effective carrier, e.g., any formulation or composition capable of effectively delivering the component gene to cells in vivo. Approaches include insertion of the gene in viral vectors, including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered naked or with the help of, for example, cationic liposomes (lipofectamine) or derivatized (e.g., antibody conjugated), polylysine conjugates, gramacidin S, artificial viral envelopes or other such intracellular carriers, as well as direct injection of the gene construct or CaPO₄ precipitation carried out in vivo.

One approach for in vivo introduction of nucleic acid into a cell is by use of a viral vector containing the nucleic acid. Infection of cells with a viral vector has the advantage that a large proportion of the targeted cells can receive the nucleic acid. Additionally, molecules encoded within the viral vector are expressed efficiently in cells that have taken up viral vector nucleic acid.

Retrovirus vectors and adeno-associated virus vectors can be used as a recombinant gene delivery system for the transfer of exogenous polynucleotides in vivo, particularly into humans. These vectors provide efficient delivery of polynucleotides into cells, and the transferred polynucleotides are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, 1990). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al. (supra), and other standard laboratory manuals. Examples of suitable retroviruses include PLJ, pZIP, pWE and pEM which are known to those skilled in the art. Examples of suitable packaging virus lines for preparing both ecotropic and amphotropic retroviral systems include ψCrip, ψCre, ψ2 and ψAm.

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle (see, for example, Rosenfeld et al., 1992). Suitable adenoviral vectors derived from the adenovirus strain Ad type 5 dl324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., 1992). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors.

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. It is also one of the few viruses that may integrate its DNA into non-dividing cells, and exhibits a high frequency of stable integration. Vectors containing as little as 300 base pairs of AAV can be packaged and can integrate.

In addition to viral transfer methods, such as those illustrated above, non-viral methods can also be employed to cause expression of a nucleic acid compound described herein in a cell of a subject. Typically non-viral methods of gene transfer rely on the normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described by Meuli et al. (2001) and Tam et al. (2000).

In some embodiments, a polynucleotide described herein is entrapped in liposomes bearing positive charges on their surface (e.g., lipofectins), which can be tagged with antibodies against cell surface antigens of the target cells.

In clinical settings, the gene delivery systems for the therapeutic polynucleotide can be introduced into a subject by any of a number of methods, each of which is familiar in the art. For instance, a pharmaceutical preparation of the polynucleotide delivery system can be introduced systemically, e.g., by intravenous injection. In other embodiments, initial delivery of the recombinant gene is more limited, with introduction into the subject being quite localized. For example, the gene delivery vehicle can be introduced by catheter or by stereotacetic injection.

Genetic therapies in accordance with the present invention may involve a transient (temporary) presence of the gene therapy polynucleotide in the patient or the permanent introduction of a polynucleotide into the patient.

Genetic therapies, like the direct administration of compounds discussed herein, in accordance with the present invention may be used alone or in conjunction with other therapeutic modalities.

Methods of screening test compounds are described which can identify a compound that reduces the level of Bim, PUMA and/or Bax activity in a cell of the subject.

Inhibitors of Bim, PUMA and/or Bax activity are screened by resort to assays and techniques useful in identifying drugs capable of binding to Bim, PUMA and/or Bax and thereby inhibiting biological activity in a cell, such as a pancreatic β cell, a Vasa nervorum cell, a proximal tubular epithelial cell, a renal glomerulus cell, a renal mesangial cell, a retinal capillary cell, or a progenitor cell of any one or more thereof. Such assays include the use of mammalian cell lines (for example, CHO cells or 293T cells) for phage display for expressing the Bim, PUMA and/or Bax polypeptide, and using a culture of transfected mammalian or E. coli or other microorganism to produce the proteins for binding studies of potential binding compounds.

Other conventional drug screening techniques are employed using the proteins, antibodies or polynucleotide sequences of this invention. As one example, a method for identifying compounds which specifically bind to Bim, PUMA and/or Bax can include simply the steps of contacting a selected cell expressing Bim, PUMA and/or Bax with a test compound to permit binding of the test compound to Bim, PUMA and/or Bax and determining the amount of test compound, if any, which is bound to Bim, PUMA and/or Bax. Such a method involves the incubation of the test compound and Bim, PUMA and/or Bax immobilized on a solid support. Typically, the surface containing the immobilized compound is permitted to come into contact with a solution containing the protein and binding is measured using an appropriate detection system. Suitable detection systems are known in the art.

Methods for producing antibodies, or fragments thereof, which bind Bim, PUMA and/or Bax are described above.

Computer modeling and searching technologies permit identification of compounds that can bind Bim, PUMA and/or Bax. The three dimensional geometric structure of Bim, PUMA or Bax or the active site thereof can be determined. This can be done by known methods, including X-ray crystallography, which can determine a complete molecular structure.

Methods of computer based numerical modeling can be used to complete the structure (e.g., in embodiments wherein an incomplete or insufficiently accurate structure is determined) or to improve its accuracy. Any method recognized in the art may be used, including, but not limited to, parameterized models specific to particular biopolymers such as proteins or nucleic acids, molecular dynamics models based on computing molecular motions, statistical mechanics models based on thermal ensembles, or combined models.

The three-dimensional structure of Bim, PUMA or Bax can be used to identify antagonists or agonists through the use of computer modeling using a docking program such as GRAM, DOCK, or AUTODOCK (Dunbrack et al., 1997). Computer programs can also be employed to estimate the attraction, repulsion, and steric hindrance of a candidate compound to the polypeptide. Generally the tighter the fit (e.g., the lower the steric hindrance, and/or the greater the attractive force) the more potent the potential agonist or antagonist will be since these properties are consistent with a tighter binding constant. Furthermore, the more specificity in the design of a potential agonist or antagonist the more likely that it will not interfere with other proteins.

Initially a potential compound could be obtained, for example, using methods of the invention such as by screening a random peptide library produced by a recombinant bacteriophage or a chemical library. A compound selected in this manner could then be systematically modified by computer modeling programs until one or more promising potential compounds are identified.

Such computer modeling allows the selection of a finite number of rational chemical modifications, as opposed to the countless number of essentially random chemical modifications that could be made, and of which any one might lead to a useful agonist or antagonist. Each chemical modification requires additional chemical steps, which while being reasonable for the synthesis of a finite number of compounds, quickly becomes overwhelming if all possible modifications needed to be synthesized. Thus through the use of the three-dimensional structure and computer modeling, a large number of these compounds can be rapidly screened on the computer monitor screen, and a few likely candidates can be determined without the laborious synthesis of untold numbers of compounds.

For most types of models, standard molecular force fields, representing the forces between constituent atoms and groups, are necessary, and can be selected from force fields known in physical chemistry. Exemplary forcefields that are known in the art and can be used in such methods include, but are not limited to, the Constant Valence Force Field (CVFF), the AMBER force field and the CHARM force field. The incomplete or less accurate experimental structures can serve as constraints on the complete and more accurate structures computed by these modeling methods.

Further examples of molecular modeling systems are the CHARMm and QUANTA programs (Polygen Corporation, Waltham, Mass.). CHARMm performs the energy minimization and molecular dynamics functions. QUANTA performs the construction, graphic modeling and analysis of molecular structure. QUANTA allows interactive construction, modification, visualization, and analysis of the behaviour of molecules with each other.

Methods of identifying compounds useful for the present invention also include those described in U.S. Pat. No. 5,744,310, GB 2,326,413, US 20030175819, WO 2005/073720, WO 2007/093807, WO 2008/021484, WO 03/028443, US 2008/0027145, WO 2008/040087, US 20060183687 US 20050250724, US 2006/0084085 and US 20030059776.

The compounds used in the methods of the subject invention can be incorporated into pharmaceutical compositions. Such compositions typically include the compound and a pharmaceutically acceptable carrier. As used herein, the term “pharmaceutically acceptable carrier” includes any and all solvents (such as phosphate buffered saline buffers, water, saline) dispersion media, coatings, antibacterial and antifungal agents, isotonic and absorption delaying agents, and the like, compatible with pharmaceutical administration. Supplementary active compounds can also be incorporated into the compositions. The use of such media and agents for pharmaceutically active substances is well known in the art. Formulations (compositions) are described in a number of sources that are well known and readily available to those skilled in the art. For example, Remington's Pharmaceutical Sciences (Martin E. W., Easton Pa., Mack Publishing Company, 19th ed., 1995) describes formulations which can be used in connection with the subject invention.

A pharmaceutical composition is formulated to be compatible with its intended route of administration, e.g., local or systemic. Examples of routes of administration include parenteral, e.g., intravenous, intradermal, subcutaneous, oral (e.g., inhalation), nasal, topical, transdermal, transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL (BASF, Parsippany, N.J.) or phosphate buffered saline (PBS). In all cases, the composition must be sterile and should be fluid to the extent that easy syringability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. Isotonic agents, for example, sugars, polyalcohols such as manitol, sorbitol, sodium chloride can also be included in the composition. Prolonged absorption of the injectable compositions can be brought about by including in the composition an agent that delays absorption, such as aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the compound into a sterile vehicle, which contains a basic dispersion medium and the required other ingredients from those enumerated above. In the case of sterile powders for the preparation of sterile injectable solutions, suitable methods of preparation include vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Formulations suitable for nasal administration, wherein the carrier is a solid, include a coarse powder having a particle size, for example, in the range of about 20 to about 500 microns, which is administered in the manner in which snuff is taken, i.e., by rapid inhalation through the nasal passage from a container of the powder held close up to the nose. Suitable formulations wherein the carrier is a liquid for administration by nebulizer, include aqueous or oily solutions of the compound. For administration by inhalation, the compound(s) can also be delivered in the form of drops or an aerosol spray from a pressured container or dispenser that contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays, drops, or suppositories. For transdermal administration, the active compound(s) are formulated into ointments, salves, gels, or creams, as generally known in the art.

The pharmaceutical compositions can also be prepared in the form of suppositories (e.g., with conventional suppository bases such as cocoa butter and other glycerides) or retention enemas for rectal delivery.

In another embodiment, the compound is formulated in liposomes. Such formulations can enhance cellular uptake of the compound. Liposomes containing the compound can be prepared by methods known in the art, such as described in U.S. Pat. No. 4,485,045, U.S. Pat. No. 4,544,545 and U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes can be extruded through filters of defined pore size to yield liposomes with the desired diameter.

In accordance with the invention, treatment of a subject with a therapeutically effective amount of the compound can include a single treatment or can include a series of treatments. The compounds can be administered on any appropriate schedule, e.g., from one or more times per day to one or more times per week; including once every other day, for any number of days or weeks, e.g., 1 day, 2 days, 3 days, 4 days, 5 days, 6 days, 1 week, 10 days, 2 weeks, 3 weeks, 4 weeks, 5 weeks, 6 weeks, 7 weeks, 8 weeks, 2 months, 3 months, 6 months, or more, or any variation thereon. The skilled artisan will appreciate that certain factors may influence the dosage and timing required to effectively treat a subject, including but not limited to the severity of the disease or disorder, previous treatments, the general health and/or age of the subject, and other diseases present.

The compound(s) used in the compositions and methods of the invention can be used in the form of salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include citric acid, lactic acid, tartaric acid, fatty acids, and the like. Salts may also be formed with bases. Such salts include salts derived from inorganic or organic bases, for example alkali metal salts such as magnesium or calcium salts, and organic amine salts such as morpholine, piperidine, dimethylamine or diethylamine salts.

The method for treating and/or preventing a disease associated with glucose toxicity of the invention can be combined with other suitable therapies such as insulin secretagogues (for example, SFUs and glinides), insulin sensitizers (for example, metformin), incretin mimetics (such as GLP-1, GIP and DPP-4 inhibitors) and insulin.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### Materials and Methods

Mice

IL-1 receptor (IL-1R^{-/-}) deficient mice were obtained from Dr. M. Labow (Roche) and backcrossed from the original mixed 129SV/C57BL/6 background onto the C57BL/6 genetic background for 8 generations. C57BL/6 (wt) mice and Fas deficient B6^{lpr/lpr} mice on a C57BL/6 genetic background were obtained from the Walter and Eliza Hall Institute animal breeding facility (Kew, Victoria, Australia). Mice deficient in the BH3-only proteins bim, puma, noxa, bid and mice deficient in bax or bak have been previously described (Bouillet et al., 1999; Villunger et al., 2003; Kaufmann et al., 2007: Knudson et al., 1995; Lindsten et al., 2000). The Bad knockout mice were kindly provided by Nika Danial (Dana-Farber Cancer Institute Boston, Mass., USA) (Ranger et al., 2003).). Puma-, Noxa- and Bid-deficient mice were generated on an inbred C57BL/6 genetic background using C57BL/6-derived ES cells. Bim- and Bad deficient were originally generated on a mixed C57BL/6×129SV genetic background, using 129SV-derived ES cells, but were backcrossed with C57BL/6 mice for >20 or >8 generations prior to use in the experiments shown here. Homozygous H-2^{bml} RIP-Bcl-2 transgenic mice which express human Bcl-2 in β cells under control of the rat insulin promoter have previously been described (Allison et al., 2000). All animal experiments were approved by the institutional animal ethics committee.

Reagents

Recombinant murine IFNγ was obtained from Genentech (South San Francisco, Calif., USA) and used at 100 U/mL. IL-1β was obtained from R&D systems (Minneapolis, Minn., USA) and was used at 10 U/mL to induce apoptosis and 1 U/mL to upregulate Fas expression. MegaFasL (APO-010) was provided by Dr M Dupuis (Apoxis, Lausanne, Switzerland) and was used at 100 nmol/L. D-glucose (used at 33.3 mM) and D-ribose (used at 50 mM) were purchased from Invitrogen (Gibco products Invitrogen Corporation, Grand Island, N.Y. USA) and Sigma-Aldrich (St Louis, Mo.) respectively. The pan-caspase inhibitor z-Val-Ala-Aspfluoromethylketone (zVAD.fmk) (Enzyme Systems Products, Livermore, Calif., USA) was used at 100 μmol/L.

Preparation of Islets

Islets of Langerhans were isolated by collagenase digestion and density gradient centrifugation as described previously (McKenzie et al., 2006). Islets were washed, hand picked and cultured overnight at 37° C. and 5% CO₂ in CMRL medium-1066 (Gibco products Invitrogen Corporation, Grand Island, N.Y. USA) supplemented with 100 U/mL penicillin, 100 μg/mL streptomycin, 2 mM glutamine and 10% foetal calf serum (FCS) (JRH Biosciences, Kans., USA) (referred to below as complete CMRL).

DNA Fragmentation Assays

Uniformly sized islets (excluding very large or necrotic islets) were handpicked into 3.5 cm Petri dishes containing 1.1 mL of complete CMRL. Islets were then cultured with the appropriate stimuli to cause DNA fragmentation. At the end of the culture period, non-attached cells and islets were transferred into polypropylene tubes and washed in PBS. Islets were then dispersed with trypsin (0.1 mg/mL bovine trypsin (Calbiochem) and 2 mmol/L EDTA in PBS) for 5 min at 37° C. Islets were mechanically dispersed using a pipette, washed in PBS, and allowed to recover in complete CMRL medium for 1 h at 37° C. in 5% CO₂. Cells were then washed in PBS and resuspended in 250 μL of hypotonic buffer containing 50 mg/mL propidium iodide (Sigma-Aldrich), 0.1% sodium citrate, and 0.1% TritonX-100 which stains nuclear DNA. The cells were then analyzed on a FACSCalibur (Becton Dickinson, Franklin Lakes, N.J., USA) using the FL3 channel. Cells undergoing apoptosis were identified by their sub-diploid DNA content as previously described (Riccardi and Nicoletti, 2006).

Nitrite Determination

Nitrite was detected in the cultures by mixing 50 μl supernatant with 50 μl Greiss reagent (Green et al., 1982). Absorbances were read at 540 nm, and nitrite concentrations were calculated using a sodium nitrite standard curve.

Flow Cytometry

Islets cells were analysed for expression of Fas by flow cytometry as previously described (Thomas et al., 1999). Islets were dispersed into single cells as described above and stained using standard procedures. Antisera used were biotinylated anti-Fas (Jo2; Pharmingen, San Diego, Calif.) followed by phycoerythrin-conjugated streptavidin (Caltag, Burlingame, Calif.). Beta cells were identified based on their high autofluorescence (particularly in the FL1 channel).

Western Blotting

Islets were incubated for 4 days with cytokines and 33.3 mM glucose, transferred to microcentrifuge tubes and washed three times in PBS. They were then resuspended in 50 μl of lysis buffer (10 mM HEPES, pH 7.4, 2 mM EDTA, 0.1% saponin, 5 mM dithiothreitol, 1 mM phenylmethylsulfonyl fluoride and 20 μg/ml leupeptin). Tubes were subjected to three cycles of freezing and thawing followed by centrifugation at 13,000 rpm for 5 min at 4° C. Lysed islets were kept at −70° C. until required. Samples were boiled for 5 min in 2× sample buffer (125 mM Tris, pH 6.8, 4% SDS, 2% 2-mercaptoethanol. 0.1% bromophenol blue and 20% glycerol). Samples were separated by SDS-PAGE and transferred to nitrocellulose using standard procedures. Western blotting was performed with anti-NOS2 Ab (Santa Cruz Biotechnology, Calif.) followed by horseradish peroxidase (HRP)-conjugated anti-rabbit Ig antibodies (Silenus Laboratories, Hawthorn, Australia) and detection with Lumi-Light Western blotting substrate (Roche Diagnostics, Mannheim, Germany). Nitrocellulose membranes were then stripped by incubation in 0.5 M NaOH for 15 minutes, washed three times in PBS and re-probed with anti-actin Ab (Santa Cruz Biotechnology, Santa Cruz, Calif.) followed by detection methods as detailed above.

Cytochrome C Release Assay

Insulin secretion assays were performed as previously described (Thomas et al., 2002). Two hundred islets were cultured in complete CMRL and ribose for 2 days, then washed three times in Krebs-Ringer bicarbonate buffer (KRB; 25 mmol/l HEPES, 115 mmol/L NaCl, 24 mmol/L NaHCO₃, 5 mmol/L MgCl₂, 2.5 mmol/L CaCl₂, pH 7.4) with 3 mmol/L glucose and 0.1% BSA. Groups of 20 islets in quadruplicate were incubated for 30 min in 200 μL KRB containing 3 mmol/L glucose under 5% CO₂/95% air with shaking at 37° C., followed by a second incubation in 200 μL KRB containing either 3 or 20 mmol/L glucose. Buffer was sampled for insulin after 30 min using a mouse insulin ELISA kit (Linco Research, Mo., USA).

Glucose-Stimulated Insulin Secretion Assay

Insulin secretion assays were performed as previously described (Thomas et al., 2002). Two hundred islets were cultured in complete CMRL and ribose for 2 days, then washed three times in Krebs-Ringer bicarbonate buffer (KRB; 25 mmol/l HEPES, 115 mmol/l NaCl, 24 mmol/l NaHCO₃, 5 mmol/l MgCl₂, 2.5 mmol/l CaCl₂, pH 7.4) with 3 mmol/l glucose and 0.1% BSA. Groups of 20 islets in quadruplicate were incubated for 30 min in 200 μl KRB containing 3 mmol/l glucose under 5% CO₂/95% air with shaking at 37° C., followed by a second incubation in 200 μl KRB containing either 3 or 20 mmol/l glucose. Buffer was sampled for insulin after 30 min using a mouse insulin ELISA kit (Linco Research, Mo., USA).

Real-Time qRT-PCR Analysis

RNA was prepared using the RNeasy Kit (Qiagen). First strand cDNA was prepared from 0.1-0.2 μg RNA using the High Capacity RNA-to-cDNA Master Mix (Applied Biosystems). Real-time PCR was performed using the ABI Prism 7900 (Applied Biosystems) and the Power SYBR Green PCR Master Mix (Applied Biosystems) in 15 μl reaction volumes. Data analyses were performed with the CT method using actin as an internal control. qRT-PCR was performed using the following forward and reverse primers (a kind gift from Drs DCS Huang and M Narita) (Table 1).

TABLE 1
Primers.

| Gene | Sense (5′ - 3′) | Antisense (5′ - 3′) |
|---|---|---|
| bak | CTCTCATCGGAGATGATATTAA | AGTATGATATCAGCCAAAAAGC |
| | CCG (SEQ ID NO: 28) | AGG (SEQ ID NO: 29) |
| bax | GGAGATGAACTGGATAGCAATA | GTTTGCTAGCAAAGTAGAAGAG |
| | TGG (SEQ ID NO: 30) | GGC (SEQ ID NO: 31) |
| bim | GAGTTGTGACAAGTCAACACAA | GAAGATAAAGCGTAACAGTTGT |
| | ACC (SEQ ID NO: 32) | AAGATAACC |
| | | (SEQ ID NO: 33) |
| puma | ATGCCTGCCTCACCTTCA | AGCACAGGATTCACAGTCTGGA |
| | TCT (SEQ ID NO: 34) | (SEQ ID NO: 35) |
| actin | TATTGGCAACGAGCGGTTC | CCATACCCAAGAAGGAAGGCT |
| | (SEQ ID NO: 36) | (SEQ ID NO: 37) |

Statistical Analysis

Analyses of data were performed using GraphPad Prism (GraphPad Sofware, San Diego, Calif., USA). All data shown as bar graphs are represented as means±SE. Data were analysed by one-way or two-way ANOVA with Bonferroni's posttest for comparison of multiple columns.

### Example 2

### High Concentrations of Glucose and Ribose Induce DNA Fragmentation, Cytochrome c Release and Loss of Insulin Secretion Capacity

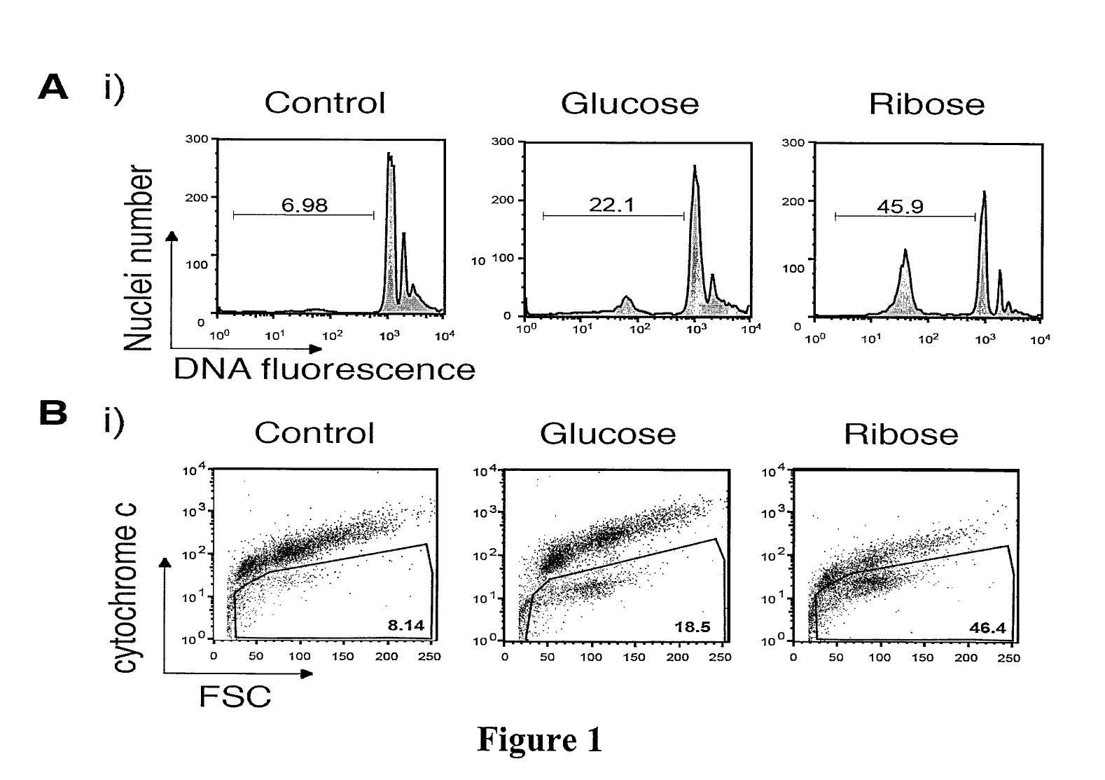

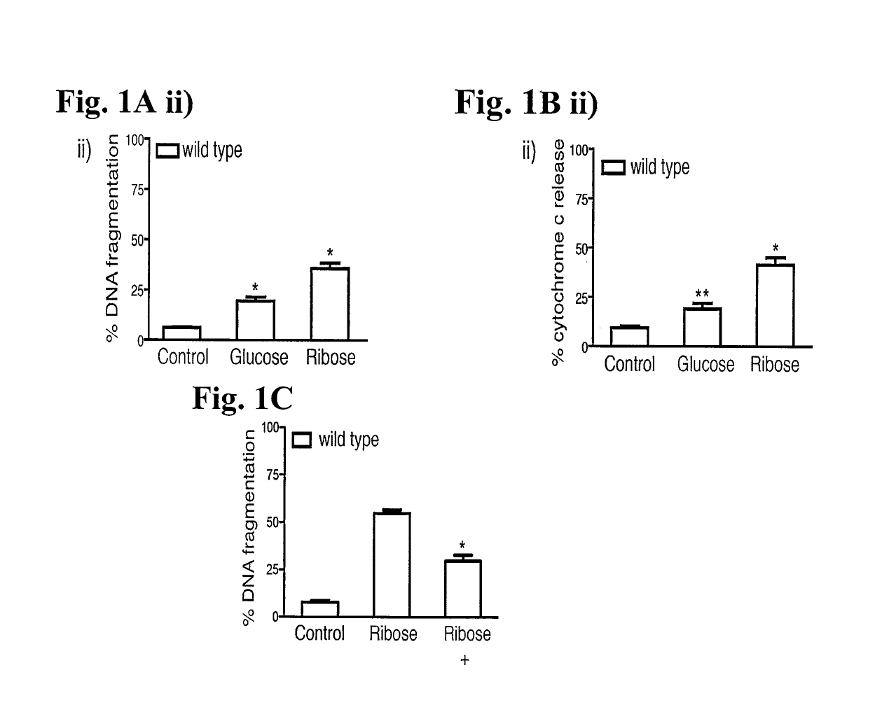

Islets from wild type C57BL/6 mice were exposed to high concentrations of reducing sugars (glucose and ribose) in vitro, and DNA fragmentation was measured. Wild-type islets cultured in high concentrations of D-glucose (33.3 mM) for 6 days displayed a modest but significant increase in DNA fragmentation compared to islets cultured in low glucose media (5.6 mM) (FIG. 1A) or cultured in 33.3 mM L-glucose (osmolarity control) (data not shown). High concentrations of ribose (50 mM), which has previously been used to mimic the effects of chronic glucose exposure over an accelerated time frame (Tanaka et al., 2002), induced significantly more DNA fragmentation (over a 4-day period) than high concentrations of glucose (FIG. 1A).

To investigate the apoptotic pathway upstream of DNA fragmentation, the present inventors studied release of cytochrome c from the mitochondrial outer membrane after 3 or 4 days of exposure to ribose and glucose respectively. High glucose concentrations resulted in a significant increase in the release of cytochrome c from the mitochondria compared to untreated islets (FIG. 1B). This increase was more pronounced when islets were cultured with high concentrations of ribose (FIG. 1B). Caspase inhibition with the pan-caspase inhibitor z-VAD.fmk significantly inhibited ribose-induced islet cell DNA fragmentation (FIG. 1C).

### Example 3

### Islets Deficient in IL-1 Receptors or Fas are not Protected from Glucose Induced DNA Fragmentation

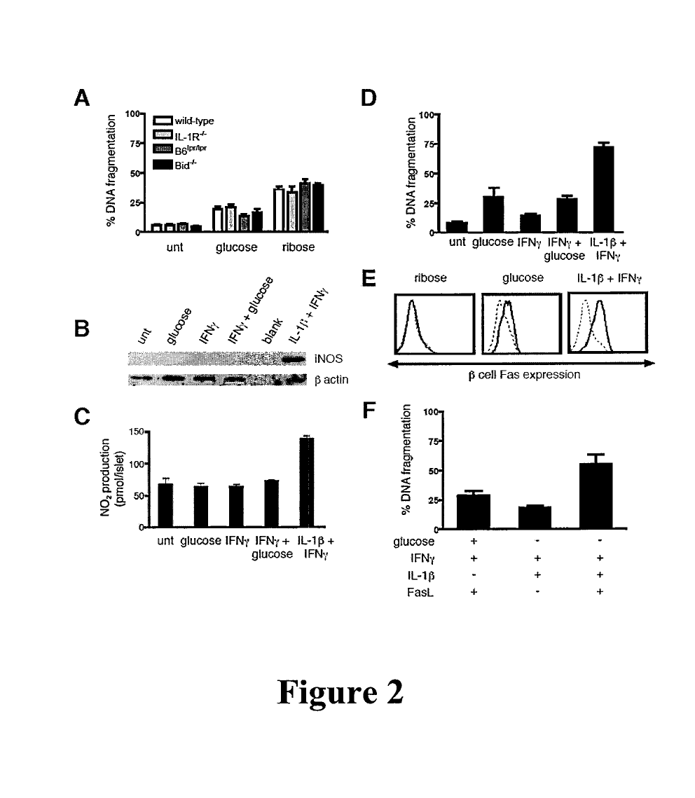

Previous studies have suggested that high glucose concentrations result in IL-1β production by beta cells and up-regulation of Fas (Maedler et al., 2001; Maedler et al., 2002). To determine the contribution of the IL-1R and the death receptor pathways to glucose/ribose induced DNA fragmentation the present inventors treated islets from mice lacking IL-1 receptors (IL-1R^{-/-}) or functional Fas (B6^{lpr/lpr}) with high concentrations of reducing sugars. DNA fragmentation was comparable to islets from wild-type mice (FIG. 2A). In addition loss of the BH3-only protein Bid, which has recently been shown to be essential for FasL-mediated islet cell apoptosis (McKenzie et al., 2008), did not protect islets from ribose or glucose toxicity (FIG. 2A). These results demonstrate that IL-1β and Fas are not essential in glucose/ribose induced DNA fragmentation.

Previous studies have shown that co-incubation of recombinant IL-1β with IFNγ leads to the induction iNOS expression, NO production and DNA fragmentation. Therefore the present inventors tested whether high glucose when co-cultured with IFNγ could induce functional concentrations of intra-islet IL-1β production by measuring iNOS expression, NO production and DNA fragmentation. The present inventors have shown that although recombinant IL-1β and IFNγ are able to induce iNOS expression, as well as a significant increase in NO production and DNA fragmentation, no iNOS expression, nor an increase in NO production and DNA fragmentation was observed in islets cultured in high glucose in the presence of IFNγ (FIG. 2 B, C, D).

The present inventors then examined Fas expression on islet beta cells after treatment with ribose or glucose. Flow cytometric analysis revealed a slight but insignificant rise in Fas expression on islet cells cultured in high glucose compared to untreated controls (FIG. 2E). However glucose-treated islets were not susceptible to FasL-induced apoptosis, even in the presence of IFNγ, which is normally required for upregulation of Fas expression on islet cells (FIG. 2F). Together these data rule out a role of both IL-1 and Fas in glucose-mediated islet toxicity in mice.

Thus, the present inventors needed to investigate other potential targets to provide a means for treating and/or preventing a disease associated with glucose toxicity in a subject.

### Example 4

### Roles of Bim, PUMA and Bax in Glucose Toxicity Induced Killing of Islet Beta Cells

Mitochondrial Cytochrome c Release and DNA Fragmentation is Prevented by Over-Expression of Bcl-2 in Beta Cells

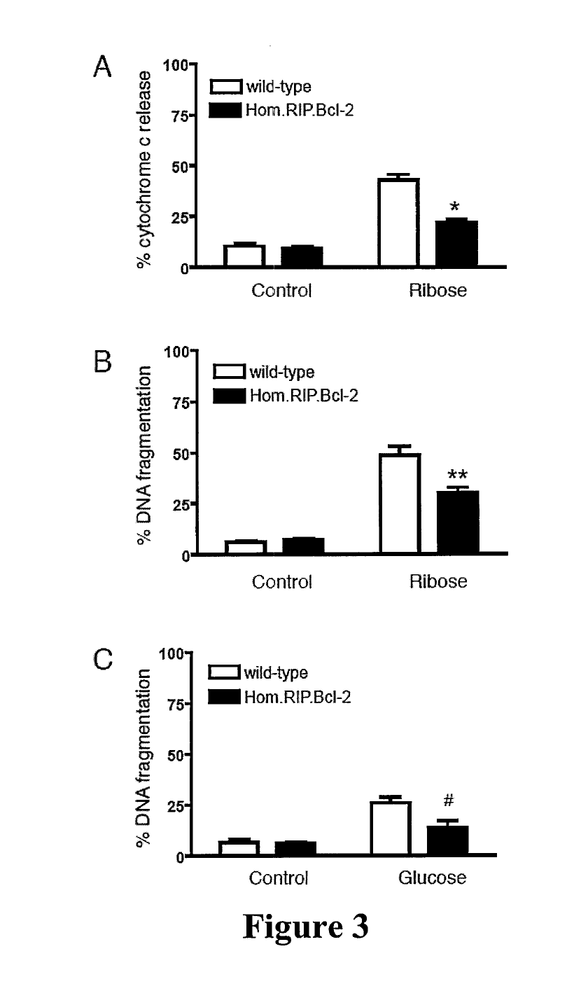

Islets over-expressing Bcl-2 in beta cells were treated with high concentrations of ribose and glucose. Over-expression of Bcl-2 significantly reduced the amount of cytochrome c released from the mitochondria and significantly prevented DNA fragmentation in response to high ribose concentrations (FIG. 3 A,B). Similarly, over-expression of Bcl-2 prevented the majority of DNA fragmentation in response to high glucose concentrations (FIG. 3C).

Individual Loss of the BH3-Only Proteins Bim or PUMA Inhibits Mitochondrial Cytochrome c Release and DNA Fragmentation

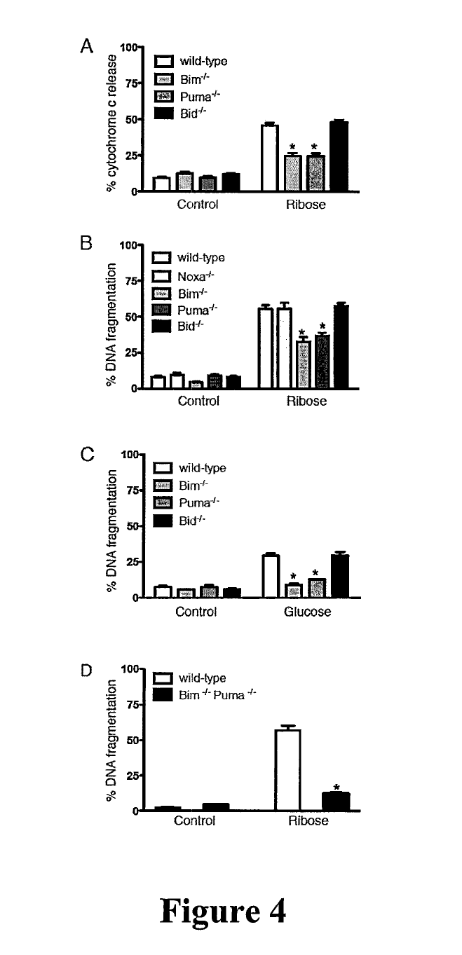

Islets were isolated from a panel of BH3-only gene knockout mice (Bad-, Bim-, Noxa-, PUMA- and Bid-deficient mice) to examine if a specific BH3-only protein is critical for this cell death. It was found that loss of either Bim or PUMA inhibited cytochrome c release from the mitochondria to a similar extent as over-expression of Bcl-2 (FIG. 4A). Similarly both Bim- and Puma-deficient knockout islets were resistant to DNA fragmentation in response to both high ribose and high glucose concentrations whereas loss of the BH3-only proteins Bad, Noxa or Bid did not protect islets (FIG. 4B, C).

Because apoptosis induced by ribose or glucose toxicity was only partially inhibited by loss of either Bim or Puma, the present inventors examined the possibility that these proteins cooperate in glucose-mediated apoptosis. Remarkably, the extent of apoptosis induced by 50 mM ribose (FIG. 4D) or 33.3 mM glucose (data not shown) in islets deficient in both Bim and Puma was not significantly above the background level observed in islets cultured in control medium, and significantly lower than apoptosis seen in islets lacking only Bim or Puma or even those overexpressing Bcl-2 (compare FIGS. 4B and 4D). These results demonstrate that Bim and Puma have critical overlapping roles in glucose toxicity induced killing of beta cells.

Loss of Bax but not Bak Prevents Ribose and Glucose Induced DNA Fragmentation

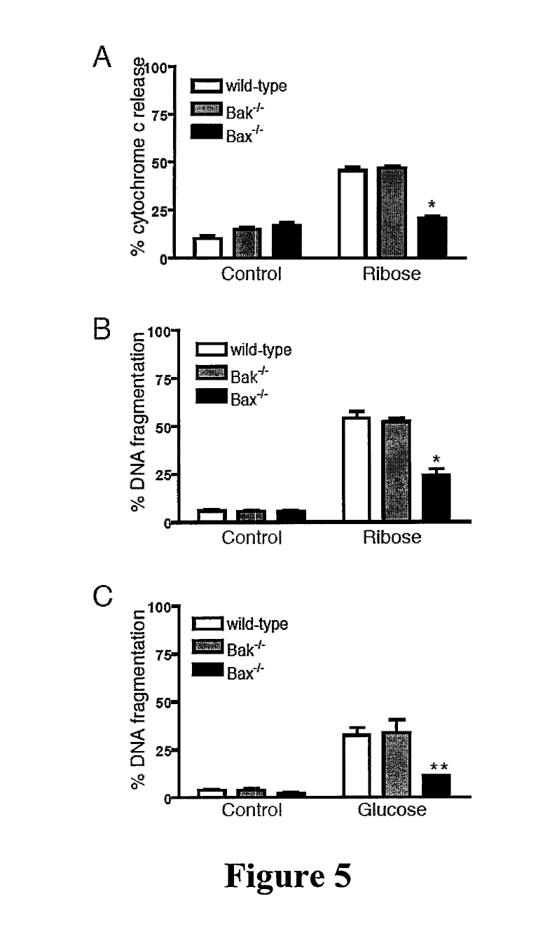

Pro-apoptotic family members Bax and Bak mediate mitochondrial dysfunction upon activation of specific BH3-only proteins. The present inventors tested whether loss of either of these proteins would result in protection of islet beta cells from apoptosis by high concentrations of ribose or glucose. Bax knockout islets were resistant to both cytochrome c release and DNA fragmentation induced by ribose, whereas islets from Bak knockout mice were as susceptible as wild-type islets (FIG. 5A, B, C). This result was surprising because usually these proteins have overlapping functions and deficiency of both is required to block apoptosis.

Loss of Bim does not Prevent the Initial Reduction in Loss of Insulin Secretory Function Induced by High Concentrations of Ribose

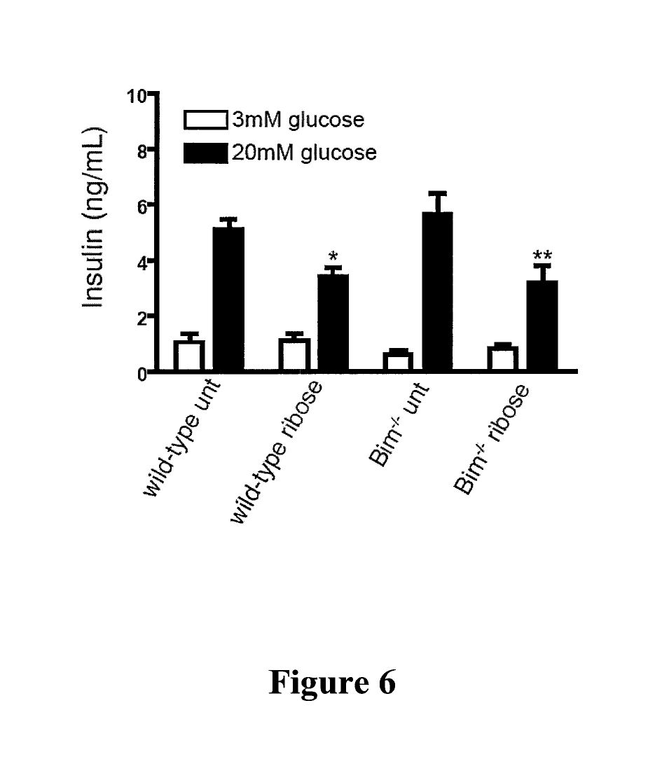

Glucose-stimulated insulin secretion assays were performed to determine the effect of high concentrations of ribose on beta cell function. Islets from either wild-type or Bim deficient mice were exposed to ribose for two days and their response glucose stimulation was measured. There was a similar significant loss of insulin secretory function in both wild type islets and Bim-deficient islets (FIG. 6). Therefore although loss of Bim provides protection from ribose and glucose-induced apoptosis, it does not prevent the initial loss of function induced by exposure to ribose.

Glucose and Ribose Toxicity Cause Up-Regulation of Puma and Bax mRNA in Islet Cells

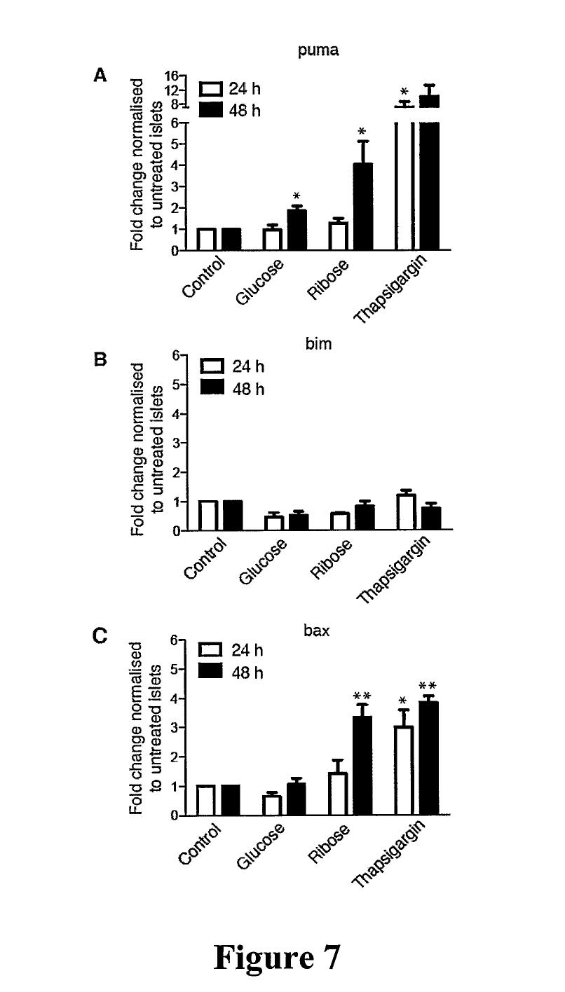

The ability of ribose or glucose to induce expression of Bim, Puma, Bax and Bak was measured by quantitative RT PCR. After 48 h exposure to 50 mM ribose, expression of puma and bax increased 4-fold (FIG. 7). Incubation with 5 μM thapsigargin, which induces apoptosis of islets, induced a 2-7-fold increase in expression of puma, bax (FIG. 7) and bak (data not shown), but not bim. Bim expression remained unchanged, even though this basal level of bim was clearly critical to cause apoptosis in response to ribose incubation.

The ER Stress Pathway Contributes to Glucose Toxicity-Induced Apoptosis of Islets

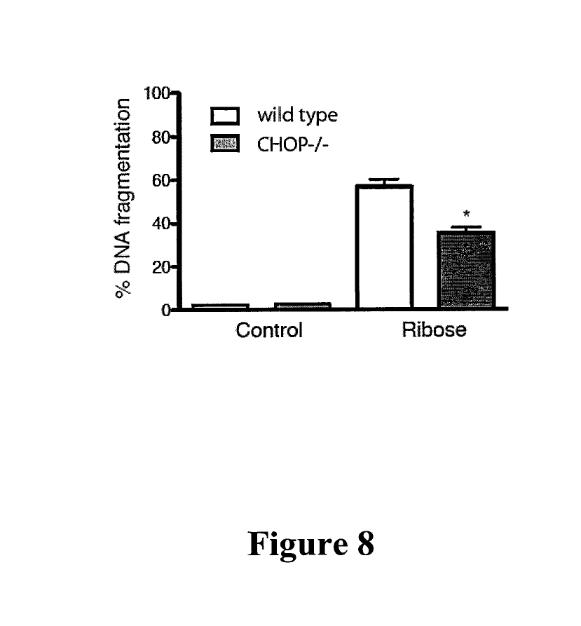

Bim was shown to be critical for ER stress-induced apoptosis in several cell types and the transcription factor CHOP was found to be critical for this process (Puthalakath et al., 2007). Because glucose-mediated apoptosis of beta cells also requires Bim, the present inventors examined the role of CHOP, and its loss indeed protected islets from ribose (FIG. 8) or glucose (data not shown) to a similar extent as loss of Bim. This result indicates that glucose toxicity appears to activate Bim in beta cells predominantly by triggering ER stress and consequent activation of its transcriptional activator CHOP (Puthalakath et al., 2007). Since no up-regulation of bim mRNA in islets exposed to high concentrations of glucose or ribose was found, the present inventors surmise that CHOP may be critical for the basal level of bim expression. This may be due to the fact that the high demand for protein synthesis and secretion place beta cells in a constant state of ER stress. However, the possibility that glucose and ribose toxicity activate Bim post-translationally cannot be excluded. The mechanisms by which glucose toxicity activates Puma are presently not clear, but they may also involve the ER stress pathway, since Puma was found to be critical for ER stress-induced apoptosis in neurons (Kieran et al., 2007, Steckley et al., 2007).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

This application claims priority from U.S. 61/086,756, the entire contents of which are incorporation herein by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Allison et al. (2000) Int Immunol 12:9-17.
Almeida and Allshire (2005) TRENDS Cell Biol15: 251-258.
Amarzguioui et al. (2004) Biochem Biophys Res Commun 316:1050-1058
Becerril et al. (1999) Biochem Biophys Res Commun 255:386-393.
Bird et al. (1988) Blood 80:1418-1422.
Bonner-Weir et al. (2000) PNAS 97:7999-8004.
Bouillet et al. (1999) Science 286:1735-1738.
Bouillet et al. (2005) Cell Death Differ 12:831-833.
Butler et al. (2003) Diabetes 52:102-110.
Calafiore (1997) Diabetes Care 20:889-896.
Chick et al. (1977) Science 197:780-782.
Docherty (1997) Clin Sci 92:321-330.
Dunbrack et al. (1997) Folding and Design, 2:R27-42.
Eizirik et al. (2008) Endocr Rev 29:42-61.
Elbashire et al. (2001) Nature 411:494-498.
Essafi et al. (2005) Oncogene 24:2317-2329.
Ewings et al. (2007) Cycle 6:2236-2240.
Gao et al. (2001) Exp Cell Res 265:145-151.
Gong et al. (2007) PLOS Medicine 4:e294.
Green et al. (1982) Anal Biochem 126:131-138.
Haseloff and Gerlach (1988) Nature 334:585-591.
Hegre et al. (1976) Acta Endocrinol Suppl (Copenh) 205:257-281.
Hemann et al. (2004) PNAS 101:9333-9338.
Huston et al. (1988) Proc Natl Acad Sci USA 85:5879-5883.
Jones et al. (1986) Nature 321:522-525.
Kaufmann et al. (2007) Cell Death Differ 14:637-639.
Kenyon et al. (1996) Diabetes Metab Rev 12:361-372.
Knudson et al. (1995) Science 270:96-99.
Kuroda et al. (2006) Proc Natl Acad Sci USA 103:14907-14912.
Letai (2008) Cancer 8: 121-132.
Lindsten et al (2000) Mol Cell 6:1389-1399.
Maedler et al. (2001) Diabetes 50:1683-1690.
Maedler et al. (2002) J Clin Invest 110:851-860.
McKenzie et al. (2006) Int Immunol 18:837-846.
McKenzie et al. (2008) Diabetes 57:1284-92.
Meuli et al. (2001) J. Invest. Dermatol. 116:131-135.
Millar and Waterhouse (2005) Funct Integr Genomics 5:129-135.
Miller (1990) Blood 76:271-278.
Morrison et al. (1984) Proc Natl Acad Sci USA 81:6851-6855.
Muoio and Newgard (2008) Nat Rev Mol Cell Biol 9:193-205
Nakano and Vousden (2001) Mol Cell 7:683-694.
O'Connor et al. (1998) EMBO J. 17:384-395.
O'Reilly et al. (2009) J Immunol 183:261-269.
Pasquinelli et al. (2005) Curr Opin Genet Develop 15:200-205.
Perriman et al. (1992) Gene 113: 157-163.
Poul et al. (2000) J Mol Biol 301:1149-1161.
Prentki and Nolan (2006) J Clin Invest 116:1802-1812.
Ranger et al. (2003) Proc Natl Acad Sci USA 100:9324-9329.
Reynolds et al. (2004) Nat Biotech 22:326-330.
Rhodes (2005) Science 307:380-384.
Riccardi and Nicoletti (2006) Nat Protoc 1:1458-1461.
Robertson (2004) J Biol Chem 279:42351-42354.
Robertson et al. (2007) FEBS Lett 581:3743-3748.
Rosenfeld et al. (1992) Cell 68:143-155.
Sandler et al. (1997) Transplantation 63:1712-1718.
Shangary and Johnson (2002) Biochem 41:9485-9495.
Shippy et al. (1999) Mol. Biotech. 12: 117-129.
Smith et al. (2000) Nature 407: 319-320.
Strasser (2005) Nat Rev Immunol 5:189-200.
Sun et al. (1986) Hybridoma 5S1:517-520.
Tam et al. (2000) Gene Ther. 7:1867-1874.
Tanaka et al. (2002) Proc Natl Acad Sci USA 99:12363-12368.
Thomas et al. (1999) J Immunol 163:1562-1569.
Thomas et al. (2002) Diabetes 51:311-316.
Tiedge et al. (1997) Diabetes 46:1733-1742.
Villunger et al. (2003) Science 302:1036-1038.
Waterhouse et al. (1998). Proc Natl Acad Sci USA 95: 13959-13964.
Waterhouse et al. (2004) Methods Mol Biol 284:307-313.
Welsh et al. (2005) Diabetes 54:3238-3244.
Willis and Adams (2005) Curr Opin Cell Biol 17:617-625.
Willis et al. (2007) Science 315:856-859.
Yu et al. (2001) Mol Cell 7:673-682.

The invention is further described by the following numbered paragraphs:

1. A method for treating and/or preventing a disease associated with glucose toxicity in a subject, the method comprising administering to the subject a compound that reduces the level of Bim, PUMA and/or Bax activity in a cell of the subject.

2. The method of paragraph 1, wherein the disease is Type 2 diabetes, Type 1 diabetes, diabetic neuropathy, diabetic nephropathy or diabetic retinopathy.

3. The method of paragraph 2, wherein the disease is Type 2 diabetes.

4. The method according to any one of paragraphs 1 to 3, wherein the compound binds Bim, PUMA and/or Bax.

5. The method of paragraph 4, wherein the compound is a polypeptide.

6. The method of paragraph 5, wherein the polypeptide is an antibody or an antigenic binding fragment thereof.

7. The method of paragraph 6, wherein the antibody or antigenic binding fragment thereof is an internalizing antibody.

8. The method of paragraph 4, wherein the compound is a fragment of Bcl-2.

9. The method according to any one of paragraphs 1 to 3, wherein the compound reduces transcription and/or translation of a gene encoding Bim, PUMA and/or Bax.

10. The method of paragraph 9, wherein the compound is a polynucleotide.

11. The method of paragraph 10, wherein the polynucleotide is selected from: an antisense polynucleotide, a sense polynucleotide, a catalytic polynucleotide, a microRNA, and a double-stranded RNA.

12. The method of paragraph 11, wherein the polynucleotide is an antisense polynucleotide which hybridises under physiological conditions to a polynucleotide comprising any one or more of the sequence of nucleotides provided as SEQ ID NOs 14 to 26.

13. The method of paragraph 11, wherein the polynucleotide is a catalytic polynucleotide capable of cleaving, under physiological conditions, a polynucleotide comprising any one or more of the sequence of nucleotides provided as SEQ ID NOs 14 to 26.

14. The method of paragraph 13, wherein the catalytic polynucleotide is a ribozyme.

15. The method of paragraph 11, wherein the polynucleotide is a double-stranded RNA (dsRNA) molecule comprising an oligonucleotide which comprises at least 19 contiguous nucleotides of any one or more of the sequence of nucleotides provided as SEQ ID NOs 14 to 26, wherein the portion of the molecule that is double-stranded is at least 19 basepairs in length and comprises said oligonucleotide.

16. The method of paragraph 15, wherein the dsRNA is expressed from a single promoter, wherein the strands of the double-stranded portion are linked by a single-stranded portion.

17. A method for treating and/or preventing a disease associated with glucose toxicity in a subject, the method comprising

i) administering cells in vitro with a compound that reduces the level of Bim, PUMA and/or Bax activity in a cell, and

ii) administering the cells from step i) into the subject.

18. The method of paragraph 17, wherein the cells are pancreatic β cells, Vasa nervorum cells, proximal tubular epithelial cells, renal glomerulus cells, renal mesangial cells, retinal capillary cells, or progenitor cells of any one or more thereof.

19. The method of paragraph 18, wherein the cells are pancreatic β cells, or progenitor cells thereof.

20. The method according to any one of paragraphs 17 to 19 which further comprises culturing the cells before they are administered to the subject.

21. Use of a compound that reduces the level of Bim, PUMA and/or Bax activity in a cell for the manufacture of a medicament for treating and/or preventing a disease associated with glucose toxicity.

22. Use of a compound that reduces the level of Bim, PUMA and/or Bax activity in a cell as a medicament for treating and/or preventing a disease associated with glucose toxicity.

23. A method for culturing cells susceptible to exposure to high concentrations of glucose, the method comprising delivering to the cells, or progenitor cells thereof, a compound that reduces the level of Bim, PUMA and/or Bax activity in the cells.

24. A cell comprising an exogenous compound that reduces the level of Bim, PUMA and/or Bax activity, wherein an isogenic cell lacking said exogenous compound is susceptible to glucose toxicity.

25. The cell of paragraph 24 which is a pancreatic β cell, a Vasa nervorum cell, a proximal tubular epithelial cell, a renal glomerulus cell, a renal mesangial cell, a retinal capillary cell, or a progenitor cell of any one or more thereof.

26. The cell of paragraph 25 which is a pancreatic β cell, or a progenitor cell thereof.

27. The cell according to any one of paragraphs 24 to 26, wherein the compound is an exogenous polynucleotide that reduces transcription and/or translation of a gene encoding Bim, PUMA and/or Bax.

28. The cell according to any one of paragraphs 24 to 27, wherein the cell is in vitro.

29. A method of identifying a compound for treating and/or preventing a disease associated with glucose toxicity, the method comprising

i) identifying a compound which binds Bim, PUMA and/or Bax,

ii) determining if the compound reduces the level of Bim, PUMA and/or Bax activity in a cell.

30. A method of identifying a compound for treating and/or preventing a disease associated with glucose toxicity, the method comprising

i) identifying a compound which hybridizes to a gene encoding Bim, PUMA and/or Bax, or a transcription product thereof, and

ii) determining if the compound reduces the production of Bim, PUMA and/or Bax in a cell,

wherein the compound is a polynucleotide.

31. A method of identifying a compound for treating and/or preventing a disease associated with glucose toxicity, the method comprising

i) identifying a compound comprising an oligonucleotide which comprises at least 19 contiguous nucleotides of any one or more of the sequence of nucleotides provided as SEQ ID NOs 14 to 26, wherein the compound comprises a double-stranded portion that is at least 19 basepairs in length which comprises said oligonucleotide, and

ii) determining if the compound reduces the production of Bim, PUMA and/or Bax in a cell,

wherein the compound is a polynucleotide.

32. The method according to any one of paragraphs 29 to 31 which comprises delivering the compound to a cell susceptible to glucose toxicity, or progenitor cell thereof, and determining if the compound enhances the survival of the cell, or progeny thereof, when compared to an isogenic cell which has not been exposed to the compound.

33. The method according to any one of paragraphs 29 to 32, wherein the cell is a pancreatic β cell, a Vasa nervorum cell, a proximal tubular epithelial cell, a renal glomerulus cell, a renal mesangial cell, a retinal capillary cell, or a progenitor cell of any one or more thereof.

34. The method of paragraph 33, wherein the cell is a pancreatic β cell or a progenitor thereof.

35. The method according to any one of paragraphs 32 to 34, wherein survival of the cell is assessed in the presence of a high sugar concentration.

36. The method of paragraph 35, wherein the sugar is glucose, ribose, fructose or a combination thereof.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A method for treating and/or preventing a disease associated with glucose toxicity in a subject, the method comprising administering to the subject a compound that reduces the level of Bim, PUMA and/or Bax activity in a cell of the subject.

2. The method of claim 1, wherein the disease is Type 2 diabetes, Type 1 diabetes, diabetic neuropathy, diabetic nephropathy or diabetic retinopathy.

3. The method of claim 1, wherein the compound binds Bim, PUMA and/or Bax.

4. The method of claim 1, wherein the compound is a small carbon based molecule.

5-16. (canceled)
